(19) United States
(12) Patent Application Publication
(10) Pub. No.: US 2026/0308652 A1
(43) Pub. Date: Oct. 8, 2026

(54) SYNTHESIS OF CAP-2 MRNA

(71) Applicant: ARIZONA BOARD OF REGENTS ON BEHALF OF THE ARIZONA STATE UNIVERSITY, Scottsdale, AZ (US)

(72) Inventors: Di LIU, Tempe, AZ (US); Hao YAN, Gilbert, AZ (US); Cong LI, Tempe, AZ (US); Xinyi TU, Tempe, AZ (US); Aleksandra PETROVA, Tempe, AZ (US)

(21) Appl. No.: 19/564,998

(22) Filed: Mar. 12, 2026

Related U.S. Application Data
(60) Provisional application No. 63/771,553, filed on Mar. 13, 2025.

(51) Int. Cl. C12P 19/34; C07H 21/02

(52) U.S. Cl. CPC C12P 19/34; C07H 21/02

(57) ABSTRACT

Described herein is method of synthesizing a Cap-2 mRNA molecule comprises co-transcriptionally installing a priming trinucleotide ppAₘAₘG to a 5′-terminus of an mRNA molecule during an in vitro transcription reaction, wherein Aₘ denotes 2′-O-methyladenosine, and adding m⁷G to the ppAₘAₘG using a vaccinia capping enzyme to yield a Cap-2 mRNA molecule comprising m⁷GpppAₘAₘG at the 5′-terminus. A trinucleotide compound having the structure ppAₘAₘG, wherein pp denotes a diphosphate moiety at a 5′-terminus, and wherein Aₘ denotes 2′-O-methyladenosine, or a pharmaceutically acceptable salt thereof, is also provided. A method of inducing an immune response in a subject comprises administering to the subject a Cap-2 mRNA molecule comprising m⁷GpppAₘAₘG at a 5′-terminus, wherein the Cap-2 mRNA molecule encodes an antigen.

### CROSS-REFERENCE TO RELATED APPLICATIONS

This application claims priority to U.S. Provisional Patent Application No. 63/771,553 filed on Mar. 13, 2025, which is incorporated by reference herein in its entirety.

### REFERENCE TO SEQUENCE LISTING

This application was filed with a Sequence Listing XML in ST.26 XML format in accordance with 37 C.F.R. § 1.831 and PCT Rule 13ter. The Sequence Listing XML file submitted in the USPTO Patent Center, “208192-0060-US02_sequence_listing_XML_5 Mar. 2026.xml,” was created on Mar. 5, 2026, contains 4 sequences, has a file size of 5.47 kilobytes (5,607 bytes), and is incorporated by reference in its entirety into the specification.

### BACKGROUND

Messenger RNA (mRNA) based therapeutics have gained tremendous interest and shown great promise in recent years, exemplified by the successful development of mRNA vaccines against infectious diseases. Compared to other genetic vectors like viruses and DNA, mRNA therapeutics offer several advantages, including transient expression with minimal risk of genomic integration, rapid and inexpensive in vitro transcription manufacturing, and the ability to transfect and be translated in a wide range of cell types, including non-dividing cells.

Eukaryotic mRNAs bear a cap structure at their 5′-termini that plays a role in translation, localization, and prevention of degradation of RNA transcripts. Naturally occurring cap structures consist of a 7-methylguanosine that is linked via a triphosphate bridge to the 5′-end of the first transcribed nucleotide, resulting in m⁷G(5′)ppp(5′)N, where N is any nucleotide. These structures are classified as Cap0 (m⁷GpppN), Cap1 (m⁷GpppNₘ), or Cap2 (m⁷GpppNₘNₘ) based on the methylation pattern.

The mRNA cap plays a role in gene expression by protecting mRNAs from degradation by exonucleases, enabling transport of RNAs from the nucleus to the cytoplasm, and participating in assembly of the translation initiation complex. In the cell, the cap is added in the nucleus and is catalyzed by multiple enzymes including RNA triphosphatase, guanylyl transferase, and methyltransferases.

Cap2 structures, which feature 2′-O-methylation at the first two nucleotides, more closely mimic the native state of mature mRNAs in higher eukaryotes compared to Cap0 and Cap1 structures. Cap2 mRNA may provide protection against innate immune recognition by cellular sensors such as RIG-1 and IFIT proteins. Additionally, Cap2 mRNA may demonstrate enhanced stability and potentially improved translation efficiency compared to Cap0 or Cap1 mRNAs.

While Cap0 and Cap1 structures are common in synthetic mRNA applications, studies comparing the translation efficiency of Cap2 versus Cap1 mRNAs have yielded varying results, likely due to variations in synthesis efficiency. Current methods for synthesizing Cap2 mRNAs face challenges in achieving high capping efficiency and purity.

What is needed are compositions and methods for synthesizing Cap-2 mRNA molecules with high efficiency and purity for use in therapeutic applications including vaccines and gene therapy.

### SUMMARY

One embodiment described herein is a method of synthesizing a Cap-2 mRNA molecule, comprising: co-transcriptionally installing a priming trinucleotide ppAₘAₘG at a 5′-terminus of an mRNA molecule during an in vitro transcription reaction, wherein Aₘ denotes 2′-O-methyladenosine; and adding m⁷G to the ppAₘAₘG using a vaccinia capping enzyme to yield a Cap-2 mRNA molecule comprising m⁷GpppAₘAₘG at the 5′-terminus. In one aspect, the in vitro transcription reaction comprises a T7 RNA polymerase and a DNA template comprising a T7 promoter sequence. In another aspect, the DNA template further comprises a poly(A) encoding region. In another aspect, the DNA template comprises a C+1T mutation on a template strand to initiate transcription with adenosine at a +1 position. In another aspect, the method further comprises purifying the mRNA molecule by lithium chloride precipitation prior to adding the m⁷G using the vaccinia capping enzyme. In another aspect, the vaccinia capping enzyme catalyzes: removal of a terminal phosphate from a 5′-diphosphate terminus of the mRNA molecule; transfer of a guanosine monophosphate moiety to a resulting 5′-monophosphate terminus; and methylation of an N7 position of a guanine base to form the m⁷GpppAₘAₘG structure. In another aspect, the adding m⁷G comprises contacting the mRNA molecule with the vaccinia capping enzyme in the presence of S-adenosylmethionine and guanosine triphosphate. In another aspect, the guanosine triphosphate is 3′-O-methylguanosine triphosphate, and wherein the Cap-2 mRNA molecule comprises m₂^{7,3}′OGpppAₘAₘG at the 5′-terminus. In another aspect, a capping efficiency of the Cap-2 mRNA molecule is at least 95%. In another aspect, the capping efficiency of the Cap-2 mRNA molecule is at least 99%. In another aspect, the in vitro transcription reaction further comprises N1-methylpseudouridine triphosphate, and wherein the Cap-2 mRNA molecule comprises N1-methylpseudouridine nucleobases. In another aspect, the method further comprises purifying the Cap-2 mRNA molecule to remove the vaccinia capping enzyme and salts following the adding m⁷G. In another aspect, the method further comprises removing double-stranded RNA contaminants from the Cap-2 mRNA molecule by cellulose purification. In another aspect, the priming trinucleotide ppAₘAₘG has a purity of at least 95%. In another aspect, the priming trinucleotide ppAₘAₘG has a purity of at least 98%. In another aspect, the Cap-2 mRNA molecule encodes a therapeutic protein or an antigen. In another aspect, the method further comprises formulating the Cap-2 mRNA molecule in a lipid nanoparticle.

Another embodiment described herein is a trinucleotide compound having a structure of ppAₘAₘG, wherein pp denotes a diphosphate moiety at a 5′-terminus, Aₘ denotes 2′-O-methyladenosine, and G denotes guanosine, or a pharmaceutically acceptable salt thereof. In one aspect, the trinucleotide compound has a purity of at least 95% as determined by high-performance liquid chromatography. In another aspect, the trinucleotide compound has a purity of at least 98%. In another aspect, the guanosine comprises a 2′-hydroxyl group and a 3′-hydroxyl group on a ribose sugar.

Another embodiment described herein is a Cap-2 mRNA molecule, comprising: a 5′ cap structure comprising m⁷GpppAₘAₘG, wherein Aₘ denotes 2′-O-methyladenosine; and a coding sequence encoding a protein of interest. In one aspect, the protein of interest is a therapeutic protein. In another aspect, the therapeutic protein is an antigen. In another aspect, the antigen is a tumor antigen or a neoantigen. In another aspect, the antigen is derived from an infectious agent. In another aspect, the Cap-2 mRNA molecule further comprises a 3′ poly(A) tail. In another aspect, the Cap-2 mRNA molecule comprises N1-methylpseudouridine nucleobases. In another aspect, the Cap-2 mRNA molecule is formulated in a lipid nanoparticle. In another aspect, the lipid nanoparticle comprises an ionizable lipid, a helper lipid, cholesterol, and a polyethylene glycol-conjugated lipid. In another aspect, the 5′ cap structure comprises m₂^{7,3′}-OGpppAₘAₘG, wherein the m₂^{7,3}′-OG denotes a 7-methylguanosine with a 3′-O-methyl modification. In another aspect, the Cap-2 mRNA molecule maintains translation under interferon stress conditions.

Another embodiment described herein is a method of inducing an immune response in a subject, comprising: administering to the subject a Cap-2 mRNA molecule comprising m⁷GpppAₘAₘG at a 5′-terminus, wherein Aₘ denotes 2′-O-methyladenosine, and wherein the Cap-2 mRNA molecule encodes an antigen. In one aspect, the Cap-2 mRNA molecule is formulated in a lipid nanoparticle. In another aspect, the lipid nanoparticle comprises an ionizable lipid, a helper lipid, cholesterol, and a polyethylene glycol-conjugated lipid. In another aspect, the antigen is a tumor antigen, a neoantigen, or an antigen derived from an infectious agent. In another aspect, the antigen is a tumor antigen or a neoantigen, and wherein the method is for treating cancer. In another aspect, the administering comprises intramuscular administration, intradermal administration, or subcutaneous administration. In another aspect, the administering comprises intramuscular administration. In another aspect, the Cap-2 mRNA molecule maintains translation under interferon stress conditions in the subject.

Another embodiment described herein is a kit for synthesizing a Cap-2 mRNA molecule, comprising: a trinucleotide compound having a structure of ppAₘAₘG, wherein pp denotes a diphosphate moiety at a 5′-terminus, Aₘ denotes 2′-O-methyladenosine, and G denotes guanosine; a vaccinia capping enzyme; and instructions for co-transcriptionally installing the trinucleotide compound at a 5′-terminus of an mRNA molecule during an in vitro transcription reaction and adding m⁷G to the trinucleotide compound using the vaccinia capping enzyme to yield a Cap-2 mRNA molecule. In one aspect, the kit further comprises one or more of S-adenosylmethionine, guanosine triphosphate, and a T7 RNA polymerase.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic diagram illustrating the structure of a 7-methylguanosine cap at the 5′-end of an mRNA molecule and the enzymatic pathway for its formation.

FIG. 2 shows a workflow for a method for synthesizing Cap-2 mRNA.

FIG. 3 shows a comparison of Cap2 mRNA to existing technologies. Cap2 mRNA enables interferon-resilient translation. Type I interferons activate the interferon-stimulated response element (ISRE) program, inducing innate RNA sensors such as IFIT1 that inhibits translation of Cap1 mRNA (used in approved mRNA drugs). In contrast, Cap2 mRNA reduces IFIT1 engagement, preserves translation initiation, and sustains translation during IFN stress.

FIG. 4 shows the structure of different mRNA 5′-caps. 2′-O-methy groups at N1 (for Cap1) and at both N1 and N2 (for Cap2) are highlighted with dashed circles.

FIG. 5A-D show a comparison between the method described herein and current capping strategies. FIG. 5A-B shows the structure of CleanCap® (TriLink BioTechnologies), a Cap 1 reagent (FIG. 5A) and engineered DNAc template (FIG. 5B) for co-transcriptional capping. In some aspects, the T7 top (5′→*3′) and bottom (3′→*5′) strands comprise SEQ ID NO: 1-2, respectively. FIG. 5C shows enzymatic capping. FIG. 5D shows hybrid capping for efficient Cap2 mRNA synthesis. Dashed circles indicate the groups introduced by GTP analogs.

FIG. 6A-E show results of the synthesis of a trinucleotide pre-capping reagent, ppAₘAₘG. FIG. 6A shows the synthesis route with the yield for each step indicated. FIG. 6B shows characterization of compound 13 by HPLC (98.9% purity). FIG. 6C shows the high-resolution mass spectrum of 13 (only positive scan is shown; assignments of the major m/z peaks are indicated). FIG. 6D shows the ¹H NMR spectrum of 13 in DMSO-d₆. FIG. 6E shows the ³¹P NMR spectrum of 13 in DMSO-d₆.

FIG. 7A-C show confirmation of successful synthesis and capping of Cap2 and ₘCap2 RNA. FIG. 7A shows site-specific RNA cleavage by RNase H allows for RNA 5′ end analysis. Cleavage is guided by a 2′-O-methyl RNA-DNA chimera. FIG. 7B shows a denaturing PAGE of cleaved 5′-fragments from eGFP mRNAs with different 5′-caps. Uncapped=34 nt (from +1 to the cleavage site); Cap1 and the pre-capped transcript=35 nt (one extra nucleotide before +1); Cap2 and mCap2=36 nt (additional m⁷G added). FIG. 7C shows an immunodot-blot assay for detecting dsRNA contaminants using J2 mAb. * indicates cellulose-purified samples.

FIG. 8A-C show Cap2 and ₘCap2 maintain translation of mRNA translation in A549-Dual cells after IFNα pretreatment. FIG. 8A-B show confocal microscopy (FIG. 8A) and flow cytometry (FIG. 8B) of eGFP after transfection of different capped mRNAs with or without IFNα (5 h pretreatment; readout at 24 h, n=3). Significance was determined using unpaired two-tailed Student's t-test. Error bars, mean±s.d. FIG. 8C shows ISRE-luciferase reporter confirming IFNα responsiveness.

FIG. 9 shows a bar graph showing normalized DGLuc/CyLuc values for different mRNA cap structures measured at three time points, according to an embodiment.

FIG. 10A shows a bar graph showing chemiluminescence measurements for DGLuc and CyLuc reporters across different mRNA cap structures, according to aspects of the present disclosure. FIG. 10B shows a bar graph showing normalized DGLuc/CyLuc ratios for different mRNA cap structures across three time points, according to an embodiment.

FIG. 11 shows the synthesis of ppm⁶AₘAₘG with m⁶Am at N1. Compared with 13, the additional methyl group in the final product 15 (ppm⁶AₘAₘG) is highlighted (dashed circle).

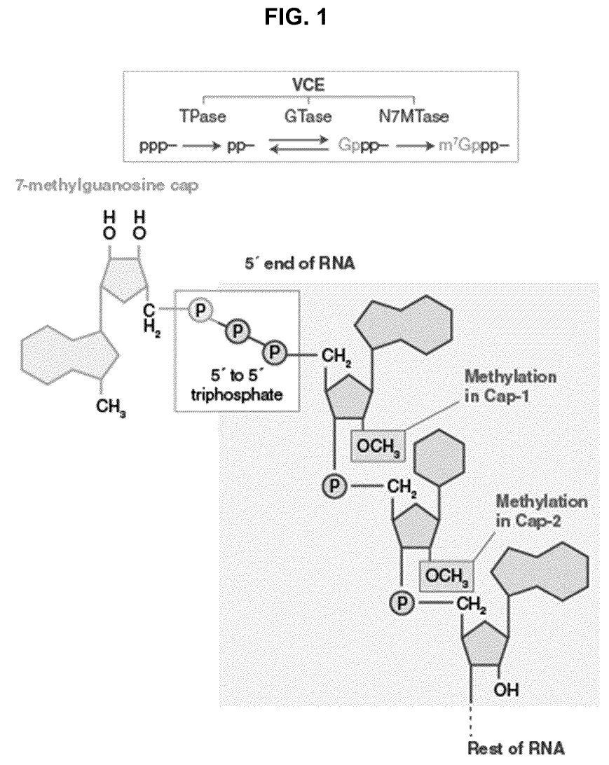

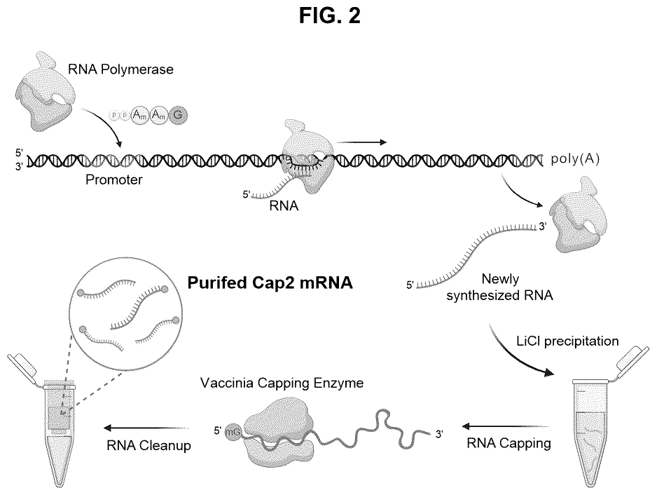

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, and chemistry described herein are well known and commonly used in the art. In case of conflict, the present disclosure, including definitions, will control the interpretation of terms. Exemplary methods and materials are described below, although methods and materials similar or equivalent to those described herein can be used in practice or testing of the embodiments and aspects described herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “-” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; −20° C., −22° C., −25° C., or −27° C., at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the term “alkyl” refers to a single bond chain of hydrocarbons ranging, in some embodiments, from 1-8 carbon atoms, and ranging in some embodiments, from 1-8 carbon atoms; examples include methyl, ethyl, propyl, isopropyl, n-butyl, sec-butyl, isobutyl, tert-butyl, pentyl, isoamyl, hexyl, octyl, and the like.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the terms “mRNA” and “mRNA molecule” are used interchangeably and refer to a single-stranded RNA molecule built up for example of A, C, G, and/or U nucleotides, i.e., nucleotides comprising adenine, guanine, cytosine, and uracil as the respective nitrogenous base. Furthermore, an mRNA molecule contains one or more coding sequences that can be used as a template during synthesis of an amino acid sequence during translation. Thus, the mRNA molecule to be purified preferably comprises a coding sequence, i.e., a sequence that can be translated into an amino acid sequence such as a protein and that comprises a start and a stop codon. The coding sequence can be a naturally occurring sequence, a partly or fully codon optimized sequence derived from the natural sequence to be used or an artificial sequence. Codon optimization refers to a technique which is applied to maximize protein expression by increasing the translational efficiency of the respective mRNA molecule as in some cases codons exist that are preferentially used by some species for a given amino acid. Further, said mRNA molecule might comprise a 5′ and/or a 3′ untranslated region (UTRs), one or more internal ribosome entry site(s) (IRES), one or more additional sequences for promoting translation, and/or one or more modifications to adjust and/or extend the duration of action. Further features of mRNA molecules are described in further detail below.

Hence, the term “mRNA” should be understood to mean any RNA molecule which is suitable for the expression of an amino acid sequence or which is translatable into an amino acid sequence such as a protein. Thus, an “mRNA molecule” is intended to be understood as an mRNA molecule that exhibits a property and/or activity by which it is characterized and thus, can be translated into an amino acid sequence such as a protein with said amino acid sequence showing the activity and/or property by which it is characterized.

Eukaryotic mRNAs bear a “cap” structure at their 5′-termini that is well known to play an important role in translation. It facilitates translation or localization, and/or prevents degradation of an RNA transcript when incorporated at the 5′-end of an RNA transcript. Naturally occurring cap structures consist of a 7-methyl guanosine that is linked via a triphosphate bridge to the 5′-end of the first transcribed nucleotide, resulting in m⁷G(5′)ppp(5′)N, where N is any nucleotide. These structures are classified as Cap0 (m⁷GpppN); Cap1 (m⁷GpppNₘ); or Cap2 (m⁷GpppNₘNₘ) based on the methylation pattern. Cap2 structure, which features 2′-O-methylation at the first two nucleotides, provides the strongest protection against innate immune recognition by cellular sensors such as RIG-1 and IFIT proteins. Additionally, Cap2 mRNA demonstrates enhanced stability and potentially improved translation efficiency compared to Cap0 or Cap1 mRNAs. The cap may include a triphosphate, a tetraphosphate or a pentaphosphate group joining the nucleotides. The mRNA cap plays an important role in gene expression. It protects the mRNAs from degradation by exonucleases, enables transport of RNAs from the nucleus to the cytoplasm, and participates in assembly of the translation initiation complex. The cap may include a triphosphate, a tetraphosphate or a pentaphosphate group joining the two nucleotides.

In the cell, the cap is added in the nucleus and is catalyzed by multiple enzymes including RNA triphosphatase, guanylyl transferase, and methyltransferases. The addition of the cap to the 5′-terminal end of RNA occurs after transcription but immediately after transcription initiation so that it is almost impossible to detect. The terminal nucleoside is always a 7-methylguanosine, which is in the reverse orientation to all the other nucleotides, i.e., 5′-Gppp5′GpNpNp. While Cap0 and Cap1 structures are common in synthetic mRNA applications, Cap2 structures more closely mimic the native state of mature mRNAs in higher eukaryotes, potentially offering advantages for therapeutic applications by further reducing immunogenicity and enhancing translation efficiency.

As used herein, the term “cap analog” refers to a structural derivative of an RNA cap that may differ by as little as a single element.

The term “prodrug” refers to a biologically inactive compound that can be metabolized in the body to produce a biologically active drug or the “prodrug” can be a biologically active compound where in addition to its inherent biological activity can be metabolized to another or even preferred biologically active drug. In certain embodiments, the prodrug can have its own biological activity that can be similar to or different from the active drug. For example, the prodrug can be an ester of epinephrine, for example, dipivefrin which is hydrolysed into epinephrine.

As used herein, the terms “Cap0,” “Cap1,” and “Cap2” refer to different methylation patterns of the mRNA 5′ cap structure. The term “Cap0” (m⁷GpppN) refers to mRNA with a 5′ cap structure consisting of a 7-methylguanosine (m⁷G) linked via a 5′-5′-triphosphate bridge to the first nucleotide of the RNA, without additional methylation. The term “Cap1” (m⁷GpppNₘ) refers to mRNA with a Cap0 structure that also contains a 2′-O-methylation at the first nucleotide following the m⁷G cap. The term “Cap2” or “Cap-2” (m⁷GpppNₘNₘ) refers to mRNA with a Cap1 structure that also contains a 2′-O-methylation at the second nucleotide following the m⁷G cap. Cap2 mRNA provides enhanced protection against innate immune recognition, improved mRNA stability, and may influence translation efficiency.

As used herein, “ppAₘAₘG” refers to a trinucleotide priming molecule consisting of a diphosphate (pp) linked to two consecutive 2′-O-methyladenosines (Aₘ) followed by a guanosine (G). This trinucleotide serves as the first nucleotides at the 5′-end of mRNA during co-transcriptional synthesis and is designed to ensure 2′-O-methylation at the first two nucleotides of the mRNA, which is a characteristic feature of Cap2 mRNA.

The term “Vaccinia Capping Enzyme” or “VCE” refers to a heterodimeric enzyme complex derived from Vaccinia virus that catalyzes the addition of a 5′ cap structure to RNA. VCE possesses three enzymatic activities: RNA 5′-triphosphatase, which removes the terminal phosphate from the 5′-triphosphate end of RNA; guanylyltransferase, which transfers a GMP moiety to the resulting 5′-diphosphate RNA; and methyltransferase, which adds a methyl group to the N⁷ position of the guanine to form the m⁷G cap structure. In one aspect, VCE is utilized to efficiently add an m7G cap to RNA molecules that already contain 2′-O-methylation at the first two nucleotides.

As used herein, “capping efficiency” refers to the percentage of RNA molecules that successfully receive a complete cap structure during the capping reaction. High capping efficiency, approaching 100% (e.g., at least 90-99%), indicates that virtually all RNA molecules in a preparation have been properly capped, which is critical for ensuring maximum stability, translation efficiency, and reduced immunogenicity of the mRNA product. Capping efficiency may be measured by various analytical methods, including gel electrophoresis following site-specific RNA cleavage.

The term “tailing region” or “3′-tailing region” refers to a sequence of nucleotides added to the 3′-end of an mRNA molecule after transcription. Examples of tailing regions include: a poly(A) tail, which is a sequence of adenine nucleotides, a poly(C) tail, which is a sequence of cytosine nucleotides, or a structured 3′-UTR sequence that enhances stability or translation of an “open reading frame sequence.”

The term “open reading frame” or “coding region,” as used herein, refers to any nucleic acid sequence encoding a non-regulatory protein (also referred to herein as a “protein of interest”).

The term “neoantigen” or “neoantigenic” means a class of tumor antigens that arises from a tumor-specific mutation(s) which alters the amino acid sequence of genome encoded proteins.

The term “neoplasia” means any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endothelio sarcoma, lymphangiosarcoma, lymphangio endothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lympho proliferative disorders are also considered to be proliferative diseases.

The term “neoplasia vaccine” is meant to refer to a pooled sample of neoplasia/tumor specific neoantigens, for example at least two, at least three, at least four, at least five, or more neoantigenic peptides. A “vaccine” is to be understood as meaning a composition for generating immunity for the prophylaxis and/or treatment of diseases (e.g., neoplasia/tumor). Accordingly, vaccines are medicaments which comprise antigens and are intended to be used in humans or animals for generating specific defense and protective substance by vaccination. A “neoplasia vaccine composition” can include a pharmaceutically acceptable excipient, earner or diluent.

A “dimerizing drug” refers to a small molecule that can simultaneously bind two protein molecules and induce their dimerization and activation.

As used herein, the term “skeletal muscle cell” refers to a cell that performs the function of skeletal muscle, and there is no limitation as long as the cell performs the above function, but may be fetal skeletal muscle cells or adult skeletal muscle cells.

As used herein, an “immune cell” refers to any cell comprised in the immune system. The immune cell may be a cell involved in the innate or adaptive immune response.

In some embodiments, the immune cell is a natural killer cell (NK), macrophage, or T cell. In some embodiments, the T cell is a gamma-delta T cell or a natural killer T cell (NKT cells).

“Operably-linked” refers to the association two chemical moieties so that the function of one is affected by the other, e.g., an arrangement of elements wherein the components so described are configured so as to perform their usual function.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing synthetic analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated.

Specifically, degenerate codon substitutions may be achieved by generating sequences in which the third position of one or more selected (or all) codons is substituted with mixed-base and/or deoxyinosine residues. Nucleic acids include all naturally occurring nucleosides, including all forms of nucleoside bases and furanosides found in nature. Base rings most commonly found in naturally occurring nucleosides are purine and pyrimidine rings. Naturally occurring purine rings include, for example, adenine, guanine, and N⁶-methyladenine. Naturally occurring pyrimidine rings include, for example, cytosine, thymine, 5-methylcytosine, pseudouracyl. Naturally occurring nucleosides for example include, but are not limited to, ribo, 2′-O-methyl or 2′-deoxyribo derivatives of adenosine, guanosine, cytidine, thymidine, uridine, inosine, 7-methylguanosine or pseudouridine.

The terms “nucleotide sequence,” “polynucleic acid,” or “nucleic acid sequence” refer to a sequence of bases (purines and/or pyrimidines) in a polymer of DNA or RNA, which can be single-stranded or double-stranded, optionally containing synthetic, non-natural or altered nucleotide bases capable of incorporation into DNA or RNA polymers, and/or backbone modifications (e.g., a modified oligomer, such as a morpholino oligomer, phosphorodiamate morpholino oligomer or vivo-mopholino). The terms “oligo,” “oligonucleotide” and “oligomer” may be used interchangeably and refer to such sequences of purines and/or pyrimidines. In some embodiments, the oligos can comprise chemical modifications to the internucleotide phosphate linkages and/or to the backbone sugar.

The oligonucleotides described herein may be synthesized using standard solid or solution phase synthesis methods. In certain embodiments, the oligonucleotides are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

Certain embodiments encompass isolated or substantially purified nucleic acid compositions. As used herein, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′-ends of the nucleic acid) in the genomic DNA of the organism from which the nucleic acid is derived.

The term “portion” or “fragment,” as it relates to a nucleic acid molecule, sequence or segment as used herein, when it is linked to other sequences for expression, means a sequence having at least 20 nucleotides, at least 150 nucleotides, or at least 400 nucleotides. If not employed for expressing, a “portion” or “fragment” means at least 9, at least 12, at least 15, or at least 20, consecutive nucleotides, e.g., probes and primers (oligonucleotides), corresponding to the nucleotide sequence of the nucleic acid molecules.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that is accommodated by reducing the stringency of the hybridization media to achieve the desired detection of the target nucleic acid sequence.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

As used herein, the term “derived” or “directed to” with respect to a nucleotide molecule means that the molecule has complementary sequence identity to a particular molecule of interest. As used herein, the term “derived” with respect to chemical compounds refers to the form of the compound in which a hydrogen atom is replaced with a bond to the identified moiety.

As used herein, the terms “transcription” or “transcription reaction” refers to methods for enzymatically making RNA that is complementary to a DNA template, thereby producing a number of RNA copies of a DNA sequence. The RNA molecule synthesized in a transcription reaction is referred to as a “RNA transcript,” “primary transcript” or “transcript.” Transcription reactions involving the compositions and methods provided herein employs procapped mRNA, after cell-selective removal of the TOC moiety. Transcription of a DNA template may be exponential, nonlinear or linear. A DNA template may be a double stranded linear DNA, a partially double stranded linear DNA, circular double stranded DNA, DNA plasmid, PCR amplicon, a modified nucleic acid template which is compatible with RNA polymerase.

Messenger RNA therapeutics have emerged as a transformative platform for treating a wide range of diseases, including infectious diseases and cancer. The successful development of mRNA vaccines has demonstrated the potential of mRNA-based approaches for rapid, scalable manufacturing and effective protein expression in diverse cell types. Unlike DNA-based vectors, mRNA molecules do not require entry into the nucleus for transcription and can be translated upon delivery to the cytoplasm, enabling transfection of both dividing and non-dividing cells.

Eukaryotic mRNA molecules bear a cap structure at the 5′-terminus that plays a role in translation initiation, mRNA stability, and innate immune recognition. The cap structure consists of a 7-methylguanosine linked via a 5′-5′ triphosphate bridge to the first transcribed nucleotide of the mRNA. Depending on the methylation pattern at the first and second transcribed nucleotides, cap structures may be classified as Cap0, Cap1, or Cap2. Cap0 structures lack 2′-O-methylation at the first and second transcribed nucleotides. Cap1 structures contain 2′-O-methylation at the first transcribed nucleotide. Cap2 structures contain 2′-O-methylation at both the first and second transcribed nucleotides.

In mammalian cells, Cap2 structures represent a native state of mature mRNA molecules and may provide enhanced protection against innate immune recognition by cellular sensors such as retinoic acid-inducible gene I (RIG-1) and interferon-induced protein with tetratricopeptide repeats 1 (IFIT1). Cap2 mRNA molecules may demonstrate reduced engagement by these innate immune sensors compared to Cap0 or Cap1 mRNA molecules, which may allow Cap2 mRNA to maintain translation under conditions of interferon stress.

Current methods for synthesizing Cap2 mRNA molecules face challenges in achieving high capping efficiency and purity. Co-transcriptional capping approaches using tetranucleotide cap analogs may result in low yields and heterogeneous capping due to competition between the cap analog and nucleotide triphosphates during in vitro transcription. Enzymatic capping approaches may require multiple enzymes, including cap methyltransferase 2 (CMTR2), which may be difficult to source and may not achieve complete methylation at the second transcribed nucleotide. These limitations have contributed to contradictory results in studies comparing the translation efficiency of Cap2 versus Cap1 mRNA molecules.

Described herein are compositions and methods for synthesizing Cap2 mRNA molecules with high efficiency and purity. A hybrid capping approach combines co-transcriptional priming with enzymatic cap installation. In the hybrid capping approach, a priming trinucleotide containing 2′-O-methylation at the first two nucleotide positions may be incorporated at the 5′-terminus of an mRNA molecule during in vitro transcription. The priming trinucleotide installs the 2′-O-methyl groups at the first and second transcribed nucleotide positions co-transcriptionally. Following in vitro transcription, a vaccinia capping enzyme may be used to add a 7-methylguanosine cap to the 5′-terminus of the mRNA molecule, completing the Cap2 structure.

The hybrid capping approach decouples the installation of 2′-O-methyl groups from the installation of the 7-methylguanosine cap. By using a trinucleotide priming molecule rather than a tetranucleotide cap analog, the hybrid capping approach may achieve higher priming efficiency during in vitro transcription. The vaccinia capping enzyme may catalyze cap installation with high efficiency, approaching complete capping of the mRNA molecules. The hybrid capping approach may be compatible with standard mRNA manufacturing workflows and may accommodate modified guanosine triphosphate analogs to generate Cap2 mRNA molecules with additional modifications at the cap structure.

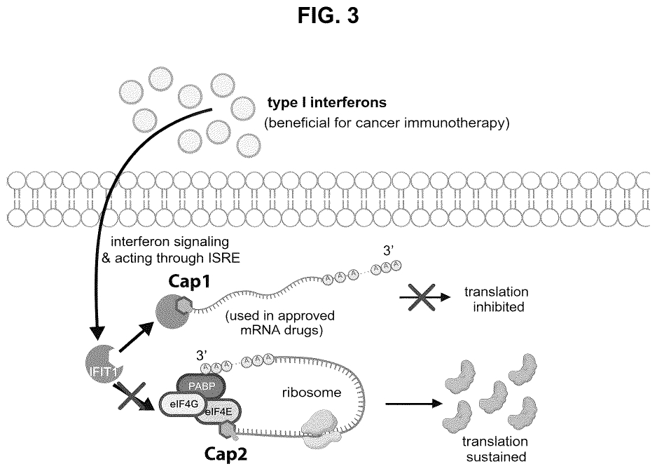

The success of mRNA vaccines has underscored the transformative potential of mRNA therapeutics, offering unparalleled opportunities for preventing and treating a wide array of diseases. Despite these advances, mRNA therapeutics face an immunogenicity-translation trade-off: exogeneous mRNA's intrinsic self-adjuvancy via innate immune sensors is useful for immunotherapy; but type I interferon (IFN) signaling suppresses translation, diminishing antigen supply. Cap-proximal 2′-O-methylation to Cap2 (2′-O-methyl at the first two transcribed nucleotides, N1 and N2) reduces engagement by retinoic-acid-inducible gene I (RIG-1) and interferon-induced protein with tetratricopeptide repeats 1 (IFIT1) relative to Cap1 (2′-O-methyl at N1 only; used in current approved mRNA drugs), suggesting a route to maintain translation under IFN (FIG. 3). However, due to the synthesis difficulty, published Cap2 results were contradictory, impeding informed deployment.

The methods and compositions described herein address these gaps by providing: A novel, efficient Cap2 mRNA synthesis method: a co-transcriptional priming trinucleotide (pre-capping reagent) installs 2′-O-methyl groups, followed by enzymatic m⁷G installation yielding high-purity Cap2 mRNA; and IFN-resilient translation: results show Cap2 sustains protein expression under IFN where matched Cap1 drops, potentially enabling self-adjuvant, unmodified IVT mRNA to preserve antigen supply during innate immune activation.

In some embodiments, the RNA (e.g., mRNA) vaccines of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the RNA (e.g., mRNA) vaccines may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partial encapsulation” or “partially encapsulated” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In some embodiments, the controlled release formulation may include, but is not limited to, tri-block co-polymers. As a non-limiting example, the formulation may include two different types of tri-block co-polymers. See Int. Pat. App. Pub. No. WO 2012131104 A1 and WO 2012131106 A1, each of which are incorporated herein by reference for such teachings.

Liposomes, lipoplexes, or lipid nanoparticles may be used to improve the efficacy of polynucleotides directed protein production as these formulations may be able to increase cell transfection by the RNA (e.g., mRNA) vaccine; and/or increase the translation of encoded protein. One such example involves the use of lipid encapsulation to enable the effective systemic delivery of polyplex plasmid DNA. See Heyes et al., Mol. Ther. 15: 713-720 (2007), which is incorporated herein by reference for such teachings. The liposomes, lipoplexes, or lipid nanoparticles may also be used to increase the stability of the polynucleotide.

In some embodiments, the RNA (e.g., mRNA) vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In some embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the RNA (e.g., mRNA) vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the RNA (e.g., mRNA) vaccine-controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the RNA (e.g., mRNA) vaccine-controlled release and/or targeted delivery formulation comprising at least one polynucleotide may comprise at least one PEG and/or PEG related polymer derivatives as described in U.S. Pat. No. 8,404,222, the contents of which are incorporated herein by reference in their entirety.

In some embodiments, the RNA (e.g., mRNA) vaccine-controlled release delivery formulation comprising at least one polynucleotide may be the controlled release polymer system described in U.S. Pat. App. Pub. No. US 20130130348 A1 which is incorporated by reference herein for such teachings.

In some embodiments, the RNA (e.g., mRNA) vaccines of the present disclosure may be encapsulated in a therapeutic nanoparticle, referred to herein as “therapeutic nanoparticle RNA (e.g., mRNA) vaccines.” Therapeutic nanoparticles may be formulated by methods described herein and known in the art such as, but not limited to, Int. Pat. App. Pub Nos. WO 2010005740 A1, WO 2010030763 A1, WO 2010005721 A1, WO 2010005723 A1, WO 2012054923 A1, U.S. Pat. App. Pub. Nos. US 20110262491 A1, US 20100104645 A1, US 20100087337 A1, US 20100068285 A1, US 20110274759 A1, US 20100068286 A1, US2 0120288541 A1, US 20130123351 A1, and US 20130230567 A1 and U.S. Pat. Nos. 8,206,747; 8,293,276; 8,318,208; and 8,318,211; each of which is incorporated by reference herein for such teachings. In some embodiments, therapeutic polymer nanoparticles may be identified by the methods described in U.S. Pat. App. Pub. No. US 20120140790 A1, which is incorporated by reference herein for such teachings.

In some embodiments, the therapeutic nanoparticle RNA (e.g., mRNA) vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present disclosure (see Int. Pat. App. Pub No. WO 2010075072 A1 and U.S. Pat. App. Pub No. US 20100216804 A1, US 20110217377 A1, and US 20120201859 A1, each of which is incorporated by reference herein for such teachings). In another non-limiting example, the sustained release formulation may comprise agents which permit persistent bioavailability such as, but not limited to, crystals, macromolecular gels and/or particulate suspensions (see U.S. Patent Publication No US 20130150295 A1, which is incorporated by reference herein for such teachings).

In some embodiments, the therapeutic nanoparticle RNA (e.g., mRNA) vaccines may be formulated to be target specific. As a non-limiting example, the therapeutic nanoparticles may include a corticosteroid (see International Pub. No. WO2011084518, the contents of which are incorporated herein by reference in their entirety). As a non-limiting example, the therapeutic nanoparticles may be formulated in nanoparticles described in Int. Pat. App. Pub No. WO 2008121949 A1, WO 2010005726 A1, WO 2010005725 A1, WO 2011084521 A1, and U.S. Pat. App. Pub No. US20100069426 A1, US20120004293 A1, and US20100104655 A1, each of which is incorporated by reference herein for such teachings.

In some embodiments, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As used herein, the term “therapeutically effective amount” means an amount of a compound described herein that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

As used herein, the term “subject” as used herein refers to humans, higher non-human primates, rodents, domestic, cows, horses, pigs, sheep, dogs and cats. In one embodiment, the subject is a human.

As used herein, the terms “treat” and “treatment” refer to both therapeutic treatment and prophylactic or preventative measures, wherein the object is to prevent or decrease an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Gastric cancer, as used herein, includes stomach cancer, which can develop in any part of the stomach and may spread throughout the stomach and to other organs; particularly the esophagus, lungs, lymph nodes, and the liver.

As used herein, the term “synergistic” as used herein refers to a therapeutic combination that is more effective than the additive effects of the two or more single agents. The combinations described herein can be evaluated in several assay systems, and the data can be analyzed utilizing a standard program for quantifying synergism, additivism, and antagonism among anticancer agents. The program utilized is that described by Chou and Talalay, in New Avenues in Developmental Cancer Chemotherapy, Academic Press (1987) Chapter 2. Combination Index values less than 0.8 indicates synergy, values greater than 1.2 indicate antagonism and values between 0.8 to 1.2 indicate additive effects. The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. In some examples, Combination effects were evaluated using both the BLISS independence model and the highest single agent (HSA) model (Leher et al. 2007, Molecular Systems Biology 3:80). BLISS scores quantify degree of potentiation from single agents and a BLISS score >0 suggests greater than simple additivity. An HSA score >0 suggests a combination effect greater than the maximum of the single agent responses at corresponding concentrations.

Response Evaluation Criteria in Solid Tumors, Version 1.1 (RECIST v1.1), can be used to evaluate tumor responses in certain human clinical trials. This section provides the definitions of the criteria used to determine objective tumor response for target lesions. “Complete response” (CR) is used to mean disappearance of all observable target lesions with pathological lymph nodes (whether target or non-target) having reduction in short axis to less than about 10 mm. “Partial response” (PR) is used to mean at least about a 30% decrease in the sum of diameters of target lesions, taking as reference the baseline sum of diameters. “Progressive disease” (PD) is used to mean at least about a 20% increase in the sum of diameters of target lesions, taking as reference the smallest sum on study (nadir), including baseline. In addition to the relative increase of about 20%, the sum also demonstrates an absolute increase of at least about 5 mm. In one example, the appearance of one or more new lesions is considered PD. “Stable disease” (SD) is used to mean neither sufficient shrinkage to qualify for PR nor sufficient increase to qualify for PD, taking as reference the smallest sum on study.

Adverse Event Grading (Severity) Scale is used to evaluate safety and tolerability with Grade 1 is mild (intervention not indicated), Grade 2 is moderate (minimal, local, or noninvasive intervention indicated), Grade 3 is severe (severe or medically significant but not immediately life threatening; hospitalization or prolongation of hospitalization indicated), Grade 4 is very severe, life threatening or disabling, urgent intervention indicated, and Grade 5 is death related to the adverse event.

### Compositions

Described herein is a composition comprising a procapped mRNA molecule and a carrier. Also described are pharmaceutical compositions and kits comprising the procapped mRNA molecules and tetrazine compounds for targeted activation for treating certain disease or conditions. In certain embodiments, the composition further comprises at least one therapeutic agent as described herein.

In certain embodiments, the composition is a pharmaceutical composition and the carrier is a pharmaceutically acceptable carrier.

In certain embodiments, the kit further comprises a procapped mRNA molecule described herein and instructions for administering the procapped mRNA molecule in combination (e.g., simultaneously or sequentially) with a mRNA encoding for a protein of interest to be expressed as part of a therapeutic or prophylactic treatment.

### Methods

The 5′-cap of eukaryotic mRNA is crucial for stability, translation initiation, and innate immune evasion. Although Cap2 mRNAs have been shown to provide the strongest protection against innate immune recognition, studies comparing the translation efficiency of Cap2 versus Cap1 mRNAs have yielded contradictory results, likely due to variations in synthesis efficiency. In some embodiments, this disclosure provides for a method for synthesizing Cap2 mRNAs. The methods described herein utilize a priming trinucleotide, ppAₘAₘG (where “Aₘ” denotes 2′-O-methyladenosine), which is efficiently installed at the 5′-end of mRNA co-transcriptionally, followed by the addition of m⁷G or an m⁷G analog using the vaccinia capping enzyme (VCE), achieving nearly 100% capping efficiency. The synthesis method described herein further enables their application in vaccines and gene therapy.

In some embodiments, the m⁷G analog can be: m⁷GTP (N7-Methylguanosine-5′-triphosphate), m¹GTP (N1-Methylguanosine-5′-triphosphate), m⁶GTP (O6-Methylguanosine-5′-triphosphate), s⁶GTP (6-Thioguanosine-5′-triphosphate), ms⁶GTP (6-Methylthioguanosine-5′-triphosphate), deaza7GTP (7-Deazaguanosine-5′-triphosphate), thienoGTP (Thienoguanosine-5′-triphosphate), oxo⁸GTP (8-Oxoguanosine-5′-triphosphate), Br⁸GTP (8-Bromoguanosine-5′-triphosphate), I⁸GTP (8-Iodoguanosine-5′-triphosphate), CI⁶GTP (2-Amino-6-chloropurineriboside-5′-triphosphate), CI6PRTP (6-Chloropurineriboside-5′-triphosphate), isoGTP (Isoguanosine-5′-triphosphate), ATP (Adenosine-5′-triphosphate), a²ATP (2-Aminoadenosine-5′-triphosphate), a²m6^{A}TP (N6-Methyl-2-aminoadenosine-5′-triphosphate), ITP (Inosine-5′-triphosphate), s⁶ITP (6-Thioinosine-5′-triphosphate), ms6|TP (6-Methylthioinosine-5′-triphosphate), XTP(Xanthosine-5′-triphosphate), CTP (Cytidine-5′-triphosphate), UTP (Uridine-5′-triphosphate), RTP (Ribavirin-5′-triphosphate), dGTP (2′-Deoxyguanosine-5′-triphosphate), d3′GTP (3′-Deoxyguanosine-5′-triphosphate), ddGTP (2′,3′-Dideoxyguanosine-5′-triphosphate), araGTP (Araguanosine-5′-triphosphate), fGTP (2′-Fluoro-2′-deoxyguanosine-5′-triphosphate), Om₂′GTP (2′-O-Methylguanosine-5′-triphosphate), Om3′GTP (3′-O-Methylguanosine-5′-triphosphate), NH22′GTP (2′-Amino-2′-deoxyguanosine-5′-triphosphate), NH23′dGTP (3′-Amino-2′,3′-dideoxyguanosine-5′-triphosphate), N32′GTP (2′-Azido-2′-deoxyguanosine-5′-triphosphate), N33′dGTP (3′-Azido-2′,3′-dideoxyguanosine-5′-triphosphate), Opp3′GTP (3′-(O-Propargyl)-GTP), Ant3′(/2′)GTP (3′(/2′)-O-Anthraniloyl-GTP), Mant3′(/2′)GTP (3′(/2′)-O—(N-methylanthraniloyl)-GTP), DTB2′GTP (2′-Desthiobiotin-GTP), DTB3′GTP (3′-Desthiobiotin-GTP), EDA2′/3′GTP (2′/3′-O-(2-Aminoethyl-carbamoyl)-guanosine-5′-triphosphate), m₂dGTP (N2-Methyl-2′-deoxyguanosine-5′-triphosphate), Ant3′(/2′)m7GTP (3′(or 2′)-(O-Anthraniloyl)-N7-methyl-GTP), or GTPαS (Guanosine-5′-O-(1-thiotriphosphate)).

In some embodiments, the methods of this disclosure can include an m⁶A modification on the first base of the mRNA. The m⁶A could be for both, Cap0, Cap1, or Cap2. The inventors have recognized that for Cap1, the CleanCap® Reagent m6AG (3′OMe) from Trilink Biotech for this purpose is not very efficient, thereby requiring more capping reagent to be included in the IVT reaction, otherwise the mRNA yield would be low.

One embodiment described herein is an mRNA comprising a Cap-2 or a composition as described herein for inducing an immune response, in a subject.

Another embodiment is the use of an mRNA comprising a Cap-2 or a composition as described herein for the manufacture of a medicament for treating a disease or disorder in a subject.

Another embodiment is the use of mRNA comprising a Cap-2 or a composition as described herein for the manufacture of a medicament for treating a disease or disorder in a subject.

Another embodiment is an mRNA comprising a Cap-2 or a composition as described herein for the prophylactic or therapeutic treatment for a disease or disorder.

In certain embodiments, the disease or disorder is a condition that requires a boost of the host immunity. In certain embodiments, the disease or disorder is a hyperproliferative disorder, such as cancer. In certain embodiments, the disease or disorder is an infectious disease.

In some embodiments, the cancer is a carcinoma, lymphoma, blastoma, sarcoma, or leukemia. In certain embodiments, the cancer is a solid tumor cancer.

In certain embodiments, the cancer is squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia, or head and neck cancer. In certain embodiments, the cancer is breast cancer.

In certain embodiments, the therapeutic agent is a therapeutic agent described herein. For example, in certain embodiments, the therapeutic agent is an immuno-stimulatory agent, a radioisotope, a chemotherapeutic drug (e.g., doxorubicin) or an immuno-therapy agent, such as antibody or an antibody fragment. In certain embodiments, the therapeutic agent is a vaccine, such as a cancer vaccine. In certain embodiments, the therapeutic agent is a tumor targeting agent, such as a monoclonal tumor-specific antibody or an aptamer. In certain embodiments, the therapeutic agent is an antibody (e.g., a monoclonal antibody, e.g., an anti-PD1 antibody). In certain embodiments, the therapeutic agent is an antigen (e.g., a tumor associated antigen or a tumor specific antigen). In certain embodiments, the therapeutic agent is a tumor antigen peptide(s).

In certain embodiments, the therapeutic agent is a chemotherapeutic agent. In certain embodiments, the chemotherapeutic agent is selected from: Abraxane (chemical name: albumin-bound or nab-paclitaxel), Adriamycin (chemical name: doxorubicin), carboplatin (brand name: Paraplatin), Cytoxan (chemical name: cyclophosphamide), daunorubicin (brand names: Cerubidine, DaunoXome), Doxil (chemical name: doxorubicin), Ellence (chemical name: epirubicin), fluorouracil (also called 5-fluorouracil or 5-FU; brand name: Adrucil), Gemzar (chemical name: gemcitabine), Halaven (chemical name: eribulin), Ixempra (chemical name: ixabepilone), methotrexate (brand names: Amethopterin, Mexate, Folex), Mitomycin (chemical name: mutamycin), mitoxantrone (brand name: Novantrone), Navelbine (chemical name: vinorelbine), Taxol (chemical name: paclitaxel), Taxotere (chemical name: docetaxel), thiotepa (brand name: Thioplex), vincristine (brand names: Oncovin, Vincasar PES, Vincrex), and Xeloda (chemical name: capecitabine). In certain embodiments, the chemotherapeutic agent is selected from: Abraxane (Paclitaxel (with albumin) Injection), Adriamycin (Doxorubicin), Afinitor (Everolimus), Alecensa (Alectinib), Alimta (PEMETREXED), Aliqopa (Copanlisib), Alkeran Injection (Melphalan), Alunbrig (Brigatinib), Aredia (Pamidronate), Arimidex (Anastrozole), Aromasin (Exemestane), Arranon (Nelarabine), Arzerra (Ofatumumab), Avastin (Bevacizumab), Bavencio (Avelumab), Beleodaq (Belinostat), Besponsa (Inotuzumab Ozogamicin), Bexxar (Tositumomab), BiCNU (Carmustine), Blenoxane (Bleomycin), Blincyto (Blinatumomab), Bosulif (Bosutinib), Braftovi (Encorafenib), Busulfex (Busulfan), Cabometyx (Cabozantinib), Calquence (Acalabrutinib), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Cinqair (Reslizumab), Clolar (Clofarabine), Cometriq (Cabozantinib), Copiktra (Duvelisib), Cosmegen (Dactinomycin), Cotellic (Cobimetinib), Cyramza (Ramucirumab), CytosarU (Cytarabine), Cytoxan (Cytoxan), Cyclophosphamide, Dacogen (Decitabine), Darzalex (Daratumumab), DaunoXome (Daunorubicin Lipid Complex), Daurismo (Glasdegib), Decadron (Dexamethasone), DepoCyt (Cytarabine Lipid Complex), Dexamethasone Intensol (Dexamethasone), Dexpak Taperpak (Dexamethasone), Docefrez (Docetaxel), Doxil (Doxorubicin Lipid Complex), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Emend (Fosaprepitant), Empliciti (Elotzumab), Erbitux (Cetuximab), Erivedge (Vismodegib), Erleada (Apalutamide), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), FloPred (Prednisolone), Fludara (Fludarabine), Folex (Methotrexate), Folotyn (Pralatrexate), FUDR (FUDR (floxuridine)), Gazyva (Obinutuzumab), Gemzar (Gemcitabine), Gilotrif (Afatinib), Gleevec (Imatinib Mesylate), Halaven (Eribulin), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Idhifa (Enasidenib), Ifex (Ifosfamide), Imbruvica (lbrutinib), Imfinzi (Durvalumab), Imlygic (Talimogene Laherparepvec), Inlyta (Axitinib), Intron A alfab (Interferon alfa-2a), Iressa (Gefitinib), Istodax (Romidepsin), Ixempra (Ixabepilone), Jakafi (Ruxolitinib), Jevtana (Cabazitaxel), Kadcyla (Ado-trastuzumab Emtansine), Keytruda (Pembrolizumab), Kisqali (Ribociclib), Kyprolis (Carfilzomib), Lanvima (Lenvatinib), Leukeran (Chlorambucil), Leukine (Sargramostim), Leustatin (Cladribine), Lorbrena (Lorlatinib), Lupron (Leuprolide), Lynparza (Olaparib), Lysodren (Mitotane), Matulane (Procarbazine), Megace (Megestrol), Mekinist (Trametinib), Mektovi (Binimetinib), Mesnex (Mesna), Mustargen (Mechlorethamine), Mutamycin (Mitomycin), Myleran (Busulfan), Mylotarg (Gemtuzumab Ozogamicin), Navelbine (Vinorelbine), Nerlynx (Neratinib), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Odomzo (Sonidegib), Oncaspar (Pegaspargase), Oncovin (Vincristine), Opdivo (Nivolumab), Panretin (Alitretinoin), Paraplatin (Carboplatin), Perjeta (Pertuzumab), Platinol (Cisplatin), PlatinolAQ (Cisplatin), Pomalyst (Pomalidomide), Portrazza (Necitumumab), Proleukin (Aldesleukin), Purinethol (Mercaptopurine), Reclast (Zoledronic acid), Revlimid (Lenalidomide), Rituxan (Rituximab), RoferonA alfaa (Interferon alfa-2a), Rubex (Doxorubicin), Rubraca (Rucaparib), Rydapt (Midostaurin), Sandostatin (Octreotide), Soltamox (Tamoxifen), Sprycel (Dasatinib), Stivarga (Regorafenib), Sutent (Sunitinib), Sylvant (Siltuximab), Synribo (Omacetaxine), Tabloid (Thioguanine), Taflinar (Dabrafenib), Tagrisso (Osimertinib), Talzenna (Talazoparib), Tarceva (Erlotinib), Targretin Capsules (Bexarotene), Tasigna (Decarbazine), Taxol (Paclitaxel), Taxotere (Docetaxel), Tecentriq (Atezolizumab), Temodar (Temozolomide), Tepadina (Thiotepa), Thioplex (Thiotepa), Tibsovo (Ivosidenib), Toposar (Etoposide), Torisel (Temsirolimus), Treanda (Bendamustine hydrochloride), Trelstar (Triptorelin), Tykerb (lapatinib), Unituxin (Dinutuximab), Valstar (Valrubicin), Varubi (Rolapitant), Vectibix (Panitumumab), Velban (Vinblastine), Velcade (Bortezomib), Venclexta (Venetoclax), Vepesid (Etoposide), Vepesid (Etoposide Injection), Verzenio (Abemaciclib), Vesanoid (Tretinoin), Vidaza (Azacitidine), Vincasar PFS (Vincristine), Vincrex (Vincristine), Vistogard (Uridine Triacetate), Vitrakvil (Larotrectinib), Vizimpro (Dacomitinib), Votrient (Pazopanib), Vumon (Teniposide), Wellcovorin IV (Leucovorin), Xalkori (Crizotinib), Xeloda (Capecitabine), Xospata (Gilteritinib), Xtandi (Enzalutamide), Yervoy (Ipilimumab), Yescarta (Axicabtagene), Yondelis (Trabectedin), Zaltrap (Ziv-aflibercept), Zanosar (Streptozocin), Zejula (Niraparib), Zelboraf (Vemurafenib), Zevalin (Ibritumomab Tiuxetan), Zoladex (Goserelin), Zolinza (Vorinostat), Zometa (Zoledronic acid) Zortress (Everolimus), Zydelig (Idelalisib), Zykadia (Ceritinib), and Zytiga (Abiraterone).

In certain embodiments, the methods of this disclosure can treat cancer in a relevant model. Mouse models for pancreatic cancer to which the methods of this disclosure are expected to demonstrate the treatment of cancer are described in Herreros-Villanueva et al., World J. Gastroenterol. 18(12): 1286-1294 (2012).

### Administration

In some embodiments, methods described herein comprise administering a mRNA comprising a Cap-2 to a subject. Such combinations may be formulated as a pharmaceutical composition and administered to a mammalian host, such as a human patient in a variety of forms adapted to the chosen route of administration, i.e., orally, parenterally, intravenously, intramuscularly, intraperitoneally, topically, or subcutaneously.

The mRNA comprising a Cap-2 may be administered intravenously or intraperitoneally by infusion or injection. Solutions of the mRNA comprising a Cap-2 or its salts is prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle is a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity is maintained, for example, by the formation of liposomes, by the maintenance of the required particle size in the case of dispersions or by the use of surfactants. The prevention of the action of microorganisms is brought about by various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions is brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions for use may be formulated using one or more physiologically acceptable carriers or excipients. Any suitable concentration of the mRNA comprising a Cap-2 may be used, and any active pharmaceutical ingredient will be administered in an amount effective to achieve its intended purpose.

Methods of making a pharmaceutical composition include admixing at least one active compound or agent, as defined above, together with one or more other pharmaceutically acceptable ingredients, such as carriers, diluents, excipients, and the like. When formulated as discrete units, such as tablets or capsule or suspension, each unit contains a predetermined amount of the active compound or agent.

Suitable formulations will depend on the method of administration. The pharmaceutical composition is preferably administered by intradermal administration, but other routes of administration include for example oral, buccal, rectal, parenteral, intramuscular, subcutaneous, intraperitoneal, transdermal, intrathecal, or nasal. The polyvalent vaccine can also be administered to the lymph nodes such as axillary, inguinal or cervical lymph nodes. The active agent may be systemic after administration or may be localized by the use of regional administration, intramural administration, or use of an implant that acts to retain the active dose at the site of implantation.

Also described are pharmaceutical compositions comprising mRNA comprising a Cap-2 for targeted delivery or activation, as well as methods of treating diseases, such as cancer or age-related diseases associated with cellular senescence, by administering such compositions to a subject in need thereof.

### Kits

Also described are kits for synthesizing a Cap-2 mRNA molecule. In one aspect the kit comprises: a trinucleotide compound having a structure of ppAₘAₘG, wherein pp denotes a diphosphate moiety at a 5′-terminus, Aₘ denotes 2′-O-methyladenosine, and G denotes guanosine; a vaccinia capping enzyme; and instructions for co-transcriptionally installing the trinucleotide compound at a 5′-terminus of an mRNA molecule during an in vitro transcription reaction and adding m⁷G to the trinucleotide compound using the vaccinia capping enzyme to yield a Cap-2 mRNA molecule. In another aspect, the kit further comprises one or more of S-adenosylmethionine, guanosine triphosphate, and a T7 RNA polymerase.

### Embodiments

One embodiment described herein is a method of synthesizing a Cap-2 mRNA molecule. The method comprises co-transcriptionally installing a priming trinucleotide ppAₘAₘG at a 5′-terminus of an mRNA molecule during an in vitro transcription reaction, wherein Aₘ denotes 2′-O-methyladenosine. The method further comprises adding m⁷G to the ppAₘAₘG using a vaccinia capping enzyme to yield a Cap-2 mRNA molecule comprising m⁷GpppAₘAₘG at the 5′-terminus.

In some aspects, the method may include one or more of the following features. The in vitro transcription reaction may comprise a T7 RNA polymerase and a DNA template comprising a T7 promoter sequence. The DNA template may further comprise a poly(A) encoding region. The DNA template may comprise a C+1T mutation on a template strand to initiate transcription with adenosine at a +1 position. The method may further comprise purifying the mRNA molecule by lithium chloride precipitation prior to adding the m⁷G using the vaccinia capping enzyme. The vaccinia capping enzyme may catalyze removal of a terminal phosphate from a 5′-diphosphate terminus of the mRNA molecule, transfer of a guanosine monophosphate moiety to a resulting 5′-monophosphate terminus, and methylation of an N7 position of a guanine base to form the m⁷GpppAₘAₘG structure. The adding m⁷G may comprise contacting the mRNA molecule with the vaccinia capping enzyme in the presence of S-adenosylmethionine and guanosine triphosphate. The guanosine triphosphate may be 3′-O-methylguanosine triphosphate, and the Cap-2 mRNA molecule may comprise m₂ 73GpppAₘAₘG at the 5′-terminus. A capping efficiency of the Cap-2 mRNA molecule may be at least 95%. The capping efficiency of the Cap-2 mRNA molecule may be at least 99%. The in vitro transcription reaction may further comprise N1-methylpseudouridine triphosphate, and the Cap-2 mRNA molecule may comprise N1-methylpseudouridine nucleobases. The method may further comprise purifying the Cap-2 mRNA molecule to remove the vaccinia capping enzyme and salts following the adding m⁷G. The method may further comprise removing double-stranded RNA contaminants from the Cap-2 mRNA molecule by cellulose purification. The priming trinucleotide ppAₘAₘG may have a purity of at least 95%. The priming trinucleotide ppAₘAₘG may have a purity of at least 98%. The Cap-2 mRNA molecule may encode a therapeutic protein or an antigen. The method may further comprise formulating the Cap-2 mRNA molecule in a lipid nanoparticle.

Another embodiment described herein is a trinucleotide compound. The trinucleotide compound has a structure of ppAₘAₘG, wherein pp denotes a diphosphate moiety at a 5′-terminus, Aₘ denotes 2′-O-methyladenosine, and G denotes guanosine, or a pharmaceutically acceptable salt thereof.

In some aspects, the trinucleotide compound may include one or more of the following features. The trinucleotide compound may have a purity of at least 95% as determined by high-performance liquid chromatography. The trinucleotide compound may have a purity of at least 98%. The guanosine may comprise a 2′-hydroxyl group and a 3′-hydroxyl group on a ribose sugar.

Another embodiment described herein is a Cap-2 mRNA molecule. The Cap-2 mRNA molecule comprises a 5′ cap structure comprising m⁷GpppAₘAₘG, wherein Aₘ denotes 2′-O-methyladenosine. The Cap-2 mRNA molecule further comprises a coding sequence encoding a protein of interest.

In some aspects, the Cap-2 mRNA molecule may include one or more of the following features. The protein of interest may be a therapeutic protein. The therapeutic protein may be an antigen. The antigen may be a tumor antigen or a neoantigen. The antigen may be derived from an infectious agent. The Cap-2 mRNA molecule may further comprise a 3′ poly(A) tail. The Cap-2 mRNA molecule may comprise N1-methylpseudouridine nucleobases. The Cap-2 mRNA molecule may be formulated in a lipid nanoparticle. The lipid nanoparticle may comprise an ionizable lipid, a helper lipid, cholesterol, and a polyethylene glycol-conjugated lipid. The 5′ cap structure may comprise m₂^{7,3}′-OGpppAₘAₘG, wherein the m₂^{7,3}′-OG denotes a 7-methylguanosine with a 3′-O-methyl modification. The Cap-2 mRNA molecule may maintain translation under interferon stress conditions.

Another embodiment described herein is a method of inducing an immune response in a subject. The method comprises administering to the subject a Cap-2 mRNA molecule comprising m⁷GpppAₘAₘG at a 5′-terminus, wherein Aₘ denotes 2′-O-methyladenosine, and wherein the Cap-2 mRNA molecule encodes an antigen.

In some aspects, the method of inducing an immune response may include one or more of the following features. The Cap-2 mRNA molecule may be formulated in a lipid nanoparticle.

The lipid nanoparticle may comprise an ionizable lipid, a helper lipid, cholesterol, and a polyethylene glycol-conjugated lipid. The antigen may be a tumor antigen, a neoantigen, or an antigen derived from an infectious agent. The antigen may be a tumor antigen or a neoantigen, and the method may be for treating cancer. The administering may comprise intramuscular administration, intradermal administration, or subcutaneous administration. The administering may comprise intramuscular administration. The Cap-2 mRNA molecule may maintain translation under interferon stress conditions in the subject.

Another embodiment described herein is a kit for synthesizing a Cap-2 mRNA molecule. In some aspects, the kit comprises: a trinucleotide compound having a structure of ppAₘAₘG, wherein pp denotes a diphosphate moiety at a 5′-terminus, Aₘ denotes 2′-O-methyladenosine, and G denotes guanosine; a vaccinia capping enzyme; and instructions for co-transcriptionally installing the trinucleotide compound at a 5′-terminus of an mRNA molecule during an in vitro transcription reaction and adding m⁷G to the trinucleotide compound using the vaccinia capping enzyme to yield a Cap-2 mRNA molecule. In another aspect, the kit further comprises one or more of S-adenosylmethionine, guanosine triphosphate, and a T7 RNA polymerase.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method of synthesizing a Cap-2 mRNA molecule, comprising:
   co-transcriptionally installing a priming trinucleotide ppAₘAₘG at a 5′-terminus of an mRNA molecule during an in vitro transcription reaction, wherein Aₘ denotes 2′-O-methyladenosine; and
   adding m⁷G to the ppAₘAₘG using a vaccinia capping enzyme to yield a Cap-2 mRNA molecule comprising m⁷GpppAₘAₘG at the 5′-terminus.
Clause 2. The method of clause 1, wherein the in vitro transcription reaction comprises a T7 RNA polymerase and a DNA template comprising a T7 promoter sequence.
Clause 3. The method of clause 1 or 2, wherein the DNA template further comprises a poly(A) encoding region.
Clause 4. The method of any one of clauses 1-3, wherein the DNA template comprises a C+1T mutation on a template strand to initiate transcription with adenosine at a +1 position.
Clause 5. The method of any one of clauses 1-4, further comprising purifying the mRNA molecule by lithium chloride precipitation prior to adding the m⁷G using the vaccinia capping enzyme.
Clause 6. The method of any one of clauses 1-5, wherein the vaccinia capping enzyme catalyzes:
   removal of a terminal phosphate from a 5′-diphosphate terminus of the mRNA molecule; transfer of a guanosine monophosphate moiety to a resulting 5′-monophosphate terminus; and
   methylation of an N7 position of a guanine base to form the m⁷GpppAₘAₘG structure.
Clause 7. The method of any one of clauses 1-6, wherein the adding m⁷G comprises contacting the mRNA molecule with the vaccinia capping enzyme in the presence of S-adenosylmethionine and guanosine triphosphate.
Clause 8. The method of any one of clauses 1-7, wherein the guanosine triphosphate is 3′-O-methylguanosine triphosphate, and wherein the Cap-2 mRNA molecule comprises m₂^{7,3}′OGpppAₘAₘG at the 5′-terminus.
Clause 9. The method of any one of clauses 1-8, wherein a capping efficiency of the Cap-2 mRNA molecule is at least 95%.
Clause 10. The method of any one of clauses 1-9, wherein the capping efficiency of the Cap-2 mRNA molecule is at least 99%.
Clause 11. The method of any one of clauses 1-10, wherein the in vitro transcription reaction further comprises N1-methylpseudouridine triphosphate, and wherein the Cap-2 mRNA molecule comprises N1-methylpseudouridine nucleobases.
Clause 12. The method of any one of clauses 1-11, further comprising purifying the Cap-2 mRNA molecule to remove the vaccinia capping enzyme and salts following the adding m⁷G.
Clause 13. The method of any one of clauses 1-12, further comprising removing double-stranded RNA contaminants from the Cap-2 mRNA molecule by cellulose purification.
Clause 14. The method of any one of clauses 1-13, wherein the priming trinucleotide ppAₘAₘG has a purity of at least 95%.
Clause 15. The method of any one of clauses 1-14, wherein the priming trinucleotide ppAₘAₘG has a purity of at least 98%.
Clause 16. The method of any one of clauses 1-15, wherein the Cap-2 mRNA molecule encodes a therapeutic protein or an antigen.
Clause 17. The method of any one of clauses 1-16, further comprising formulating the Cap-2 mRNA molecule in a lipid nanoparticle.
Clause 18. A trinucleotide compound having a structure of ppAₘAₘG, wherein pp denotes a diphosphate moiety at a 5′-terminus, Aₘ denotes 2′-O-methyladenosine, and G denotes guanosine, or a pharmaceutically acceptable salt thereof.
Clause 19. The trinucleotide compound of clause 18 wherein the trinucleotide compound has a purity of at least 95% as determined by high-performance liquid chromatography.
Clause 20. The trinucleotide compound of clause 18 or 19, wherein the trinucleotide compound has a purity of at least 98%.
Clause 21. The trinucleotide compound of any one of clauses 18-20, wherein the guanosine comprises a 2′-hydroxyl group and a 3′-hydroxyl group on a ribose sugar.
Clause 22. A Cap-2 mRNA molecule, comprising:
   a 5′ cap structure comprising m⁷GpppAₘAₘG, wherein Aₘ denotes 2′-O-methyladenosine; and
   a coding sequence encoding a protein of interest.
Clause 23. The Cap-2 mRNA molecule of clause 22, wherein the protein of interest is a therapeutic protein.
Clause 24. The Cap-2 mRNA molecule of clause 22 or 23, wherein the therapeutic protein is an antigen.
Clause 25. The Cap-2 mRNA molecule of any one of clauses 22-24, wherein the antigen is a tumor antigen or a neoantigen.
Clause 26. The Cap-2 mRNA molecule of any one of 22-25, wherein the antigen is derived from an infectious agent.
Clause 27. The Cap-2 mRNA molecule of any one of clauses 22-26, further comprising a 3′ poly(A) tail.
Clause 28. The Cap-2 mRNA molecule of any one of clauses 22-27, wherein the Cap-2 mRNA molecule comprises N1-methylpseudouridine nucleobases.
Clause 29. The Cap-2 mRNA molecule of any one of clauses 22-28, wherein the Cap-2 mRNA molecule is formulated in a lipid nanoparticle.
Clause 30. The Cap-2 mRNA molecule of any one of clauses 22-29, wherein the lipid nanoparticle comprises an ionizable lipid, a helper lipid, cholesterol, and a polyethylene glycol-conjugated lipid.
Clause 31. The Cap-2 mRNA molecule of any one of clauses 22-30, wherein the 5′ cap structure comprises m₂^{7,3}′-OGpppAₘAₘG, wherein the m₂^{7,3}′-OG denotes a 7-methylguanosine with a 3′-O-methyl modification.
Clause 32. The Cap-2 mRNA molecule of any one of clauses 22-31, wherein the Cap-2 mRNA molecule maintains translation under interferon stress conditions.
Clause 33. A method of inducing an immune response in a subject, comprising:
   administering to the subject a Cap-2 mRNA molecule comprising m⁷GpppAₘAₘG at a 5′-terminus, wherein Aₘ denotes 2′-O-methyladenosine, and wherein the Cap-2 mRNA molecule encodes an antigen.
Clause 34. The method of clause 33, wherein the Cap-2 mRNA molecule is formulated in a lipid nanoparticle.
Clause 35. The method of clause 33 or 34, wherein the lipid nanoparticle comprises an ionizable lipid, a helper lipid, cholesterol, and a polyethylene glycol-conjugated lipid.
Clause 36. The method of any one of clauses 33-35, wherein the antigen is a tumor antigen, a neoantigen, or an antigen derived from an infectious agent.
Clause 37. The method of any one of clauses 33-36, wherein the antigen is a tumor antigen or a neoantigen, and wherein the method is for treating cancer.
Clause 38. The method of any one of clauses 33-37, wherein the administering comprises intramuscular administration, intradermal administration, or subcutaneous administration.
Clause 39. The method of any one of clauses 33-38, wherein the administering comprises intramuscular administration.
Clause 40. The method of any one of clauses 33-39, wherein the Cap-2 mRNA molecule maintains translation under interferon stress conditions in the subject.
Clause 41. A kit for synthesizing a Cap-2 mRNA molecule, comprising:
   a trinucleotide compound having a structure of ppAₘAₘG, wherein pp denotes a diphosphate moiety at a 5′-terminus, Aₘ denotes 2′-O-methyladenosine, and G denotes guanosine;
   a vaccinia capping enzyme; and
   instructions for co-transcriptionally installing the trinucleotide compound at a 5′-terminus of an mRNA molecule during an in vitro transcription reaction and adding m⁷G to the trinucleotide compound using the vaccinia capping enzyme to yield a Cap-2 mRNA molecule.
Clause 42. The kit of clause 41, further comprising one or more of S-adenosylmethionine, guanosine triphosphate, and a T7 RNA polymerase.

### Examples

In the wake of the timely, successful development of mRNA vaccines for responding to the COVID-19 pandemic, mRNA therapeutics have gained enormous interest across both fundamental and translational research. Compared with viral and DNA vectors, mRNA offers several advantages: (1) mRNA expression is transient, with minimal risk of genomic integration; (2) in vitro transcription (IVT) allows for the rapid, inexpensive, cell-free manufacturing of mRNA; (3) unlike DNA, which requires entry into nucleus (typically during cell division) for transcription, mRNA can be translated upon being delivered into cytoplasm, enabling transfection of a wider range of cell types (including non-dividing cells). When mRNA encodes an antigen for expression in antigen-presenting cells (APCs, e.g., dendritic cells, DCs), it functions as a vaccine: prophylactic against infectious diseases for pathogen antigens, or therapeutic against cancers for tumor antigens, e.g., tumor-associated (TAA) or tumor-specific (TSA) antigens. However, the mRNA-based antigen delivery faces a long-standing challenge of immunogenicity-translation trade-off. Exogeneous mRNA molecules strongly activate numerous innate immune RNA sensors (or PRRs, pattern recognition receptors), and this inherent self-adjuvancy should have been desirable and exploited in cancer immunotherapy due to the often immunosuppressive tumor microenvironment (TME) and the importance of such immunostimulatory activities for promoting T cell-mediated immunity. However, activation of PRRs and the elicited type I interferon (IFN) response inevitably suppresses mRNA translation, leading to low antigen expression and negatively affecting the immune response5.

Real-world examples illustrate this tradeoff. Both Spikevax (or mRNA-1273; Moderna COVID-19 vaccine) and Comirnaty (or BNT162b2; BioNTech-Pfizer COVID-19 vaccine) use N1-methylpseudouridine (m¹ψ) modification to increase protein production and reduce inflammatory sensing and have achieved high efficacy and global approval. The 2023 Nobel Prize in Physiology or Medicine recognized Karikó and Weissman for nucleoside-base modifications that enabled these vaccines. In contrast, the COVID-19 vaccine candidate (CVnCoV) by CureVac (the earliest dedicated mRNA company) underperformed in efficacy trials and eventually failed in reaching approval. Among multiple factors implicated, the use of unmodified RNA has been widely discussed as one of the main contributors to this setback. It is now widely appreciated that for certain applications (e.g., vaccines for respiratory diseases), where high antibody titers are primary, modified mRNA is favored as it allows higher antigen expression for stronger antibody induction; whereas, for therapeutic cancer vaccines, when antibodies are less important and T-cell response is more critical, unmodified (uridine) mRNA could be advantageous due to heightened innate stimulation. Consistent with this view, all the mRNA vaccine candidates in BioNTech's current oncology pipeline employ unmodified mRNA. Therefore, an inquiry is whether or how the in vitro transcribed mRNA translation could be sustained under IFN stress. The focus here is on the mRNA 5′-cap.

Contradictory Results of IVT Cap2 mRNA

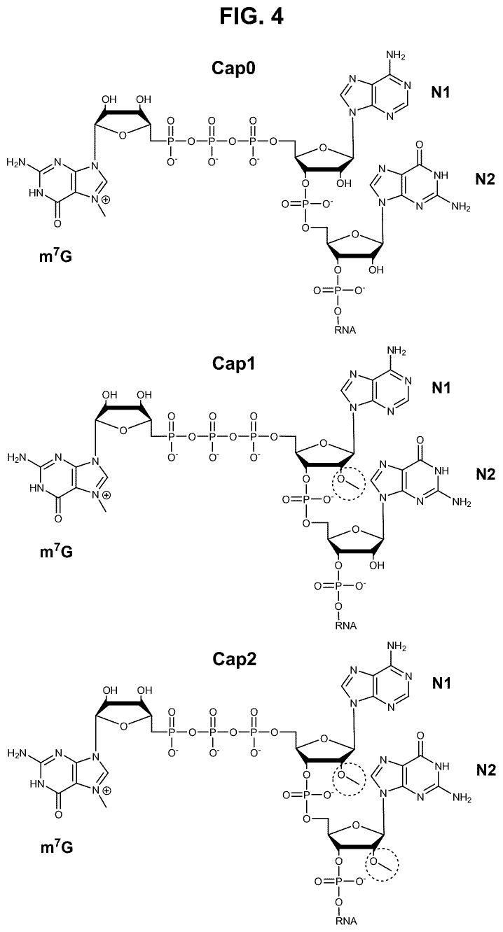

The 5′-end of eukaryotic mRNAs bears an N⁷-methylguanosin(m⁷G) cap linked by a 5′-5 triphosphate bridge, which has functions of mediating translation initiation, protecting mRNA from 5′→*3′ exonucleases, and marking self RNA to avoid innate immune sensing. Depending on whether the first (N1) and second (N2) transcribed nucleotides are 2′-O-methylated, the 5′-cap exists in different forms (FIG. 4): Cap0, if no 2′-O-methylation; Cap1, only N1 2′-O-methylation; and Cap2, N1+N2 2′-O-methylation. In mammals, N1 2′-O-methylation takes place co-transcriptionally in the nucleus; so uncapped and Cap0 RNAs could be recognized as viral/non-self RNAs. N2 2′-O-methylation is catalyzed by cap methyltransferase 2 (CMTR2) in the cytoplasm, and about half of mRNAs in human cell lines bear Cap2. Although the function of Cap2 is less clear, multiple studies implicate its role in innate recognition during viral infection. For example, RIG-1 (retinoic acid inducible gene-I), which recognizes viral dsRNA containing specific 5′-features to activate the IFN response, can weekly bind dsRNA with Cap1, but not detectably with Cap2. Furthermore, IFN signaling induces hundreds of IFN-stimulated genes (ISGs), including the IFN-induced protein with tetratricopeptide repeats (IFIT) family. The recognition of mRNA 5′-end by human IFIT1 and IFIT5 blocks the engagement of translation initiation factor eIF4E, thereby inhibiting mRNA translation. While Cap1 prevents the IFIT5 recognition, it can still be recognized by IFIT1 (K_{D} ranges from tens to hundreds of nM; Cap2 fully abolishes IFIT1 binding (FIG. 4).

Clinically, both approved COVID-19 mRNA vaccines-Comirnaty and Spikevax-use Cap1, establishing Cap1 as the current standard for mRNA medicines. The natural inquiry was whether Cap2 can enhance IVT mRNA performance; however, published data have been contradictory. Drazkowska et al., Nucleic Acids Res. 50(16): 9051-9071 (2022) (referred to as D2022 hereafter) reported that Cap2 mRNA was translated significantly lower than Cap1 (~2- to 15-fold) across all the tested cell lines. In contrast, Inagaki et al. Nat. Commun. 14(1): 2657 (2023) (referred to as 12023) reported the opposite, where Cap2 mRNA was translated ~1.5- to 4-fold of Cap1 mRNA in cells and animals. Identifying limitations (e.g., capping efficiency/purity, and downstream purification) of these prior studies, a novel, facile method for the efficient synthesis of Cap2 mRNA was developed with the hope to reconcile these contrasting results. These results differ from both D2022 and 12023: Cap1 and Cap2 mRNAs exhibit similar translation under basal conditions of the cells, but Cap1 mRNA shows substantially decreased translation under IFN stress, whereas Cap2 mRNA remains resilient. These findings motivated exploration of the utility of Cap2 mRNA for improving T-cell receptor engineered T cell (TCR-T) therapy (FIG. 3).

a Hybrid Capping Method for Cap2 mRNA Synthesis

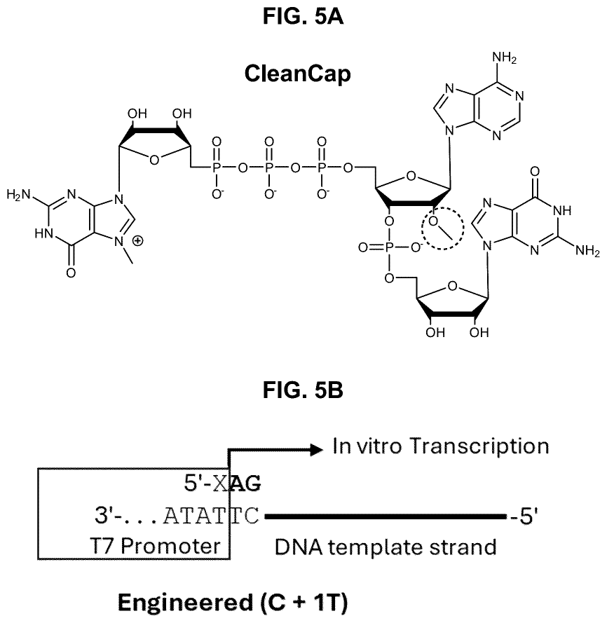

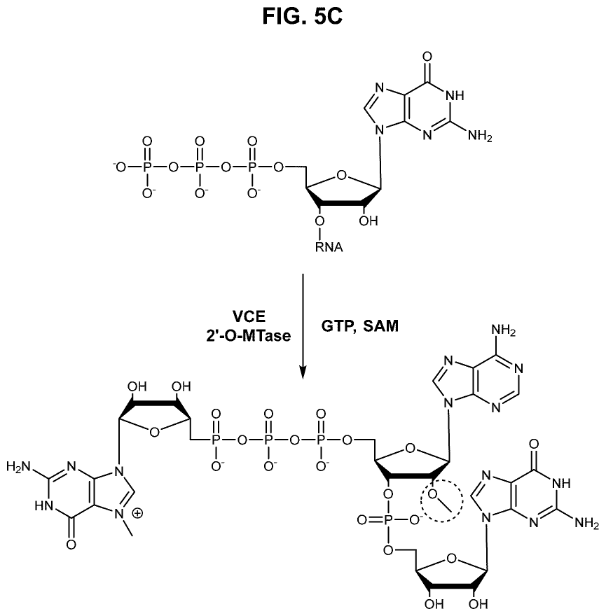

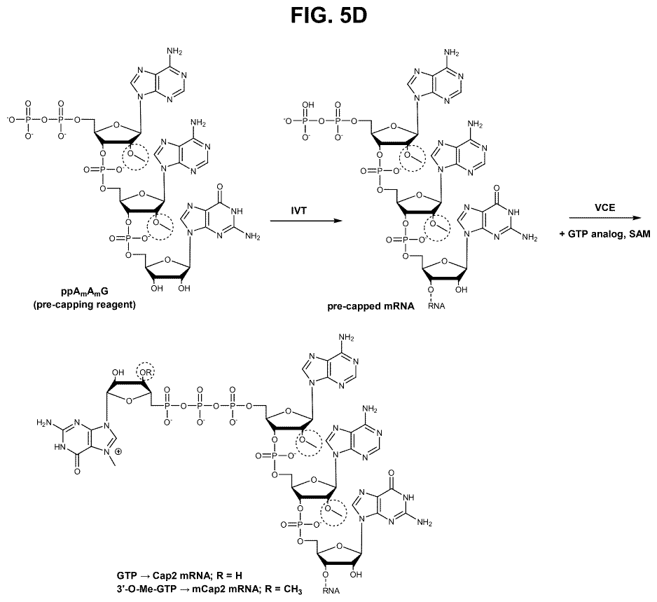

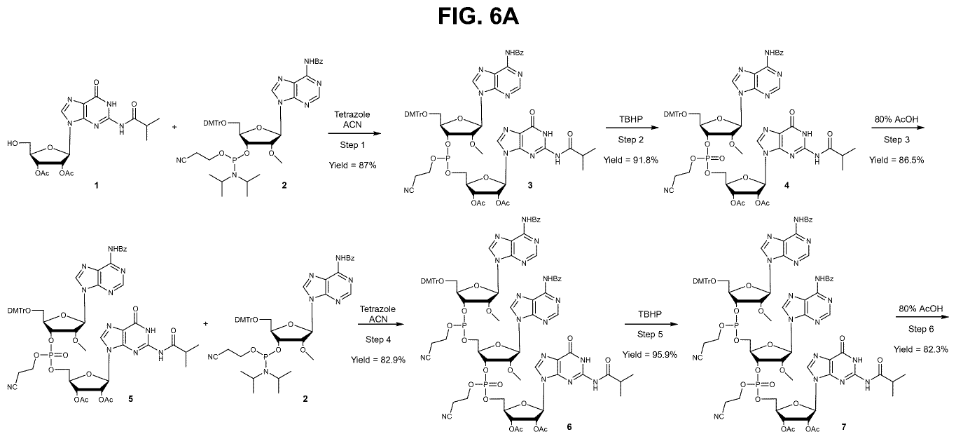

Cap1 is used by both authorized COVID-19 mRNA vaccines, but via different routes: Comirnaty employs co-transcriptional capping with TriLink's CleanCap® Cap 1 structure (FIG. 5A), which deploys a priming trinucleotide cap analog (e.g., m⁷GpppAmG) and an engineered DNA promoter to initiate with A/G at +1/+2 (i.e., a C+1T mutation on the DNA template strand, see FIG. 5B; “X” can be a variety of nucleotides or functional groups, e.g., m⁷Gppp in the CleanCap of FIG. 5A); Spikevax uses post-transcriptional enzymatic capping (FIG. 5C) with Vaccinia virus Capping Enzyme (VCE) and the mRNA cap 2′-O-methyltransferase (2′-OMTase). In some aspects, the T7 top (5′→*3′) and bottom (3′→*5′) strands comprise SEQ ID NO: 1-2, respectively (see Table 1).

TABLE 1
T7 Promoter Sequences

| Name | DNA Sequences (5′→3′) | SEQ ID NO |
|---|---|---|
| T7 Top | GTTCTAATACGACTCACTATAAG | 1 |
| T7 Bottom | CAAGATTATGCTGAGTGATATTC | 2 |

Although efficient for Cap1, Cap2 mRNA cannot be achieved through the direct adaptions of these routes. For co-transcriptional capping, because (1) Cap2 requires installing 2′-O-methyl groups on both N1 and N2, and (2) T7 RNA polymerase cannot extend a 3′-end bearing a 2′-O-methyl group, a tetranucleotide cap analog (e.g., m⁷GpppAₘGₘG or m⁷GpppAₘAₘG) should be used, as in D2022 and 12023. However, tetranucleotides prime IVT inefficiently, resulting in low yield and purities (GTP and ATP in the IVT compete with the priming tetranucleotide on the wild-type and C+1T engineered promoters, respectively; D2022 claimed high capping efficiency, which was already questioned by 12023). Uncapped or cap-heterogeneous by-products are difficult to purify. 12023 introduced a hydrophobic photocage onto the cap, enabling (1) the purification of successfully capped mRNA by RP-HPLC (reversed-phase high-performance liquid chromatography) followed by UV deprotection and (2) the removal of dsRNA contamination (detrimental for mRNA translation). However, the synthesis of photocaged cap analogs is challenging; the extra UV deprotection step adds workflow complexity; and separation is less efficient for long mRNAs. Notably, D2022 and 12023 reported opposite Cap2-vs.-Cap1 performance, underscoring the need for improved, cleaner Cap2 synthesis.

A fully enzymatic Cap2 path would require CMTR2, and key process questions remain: (1) completeness of methylation by CMTR2; (2) compatibility with VCE and 2′-OMTase in one-pot reaction; (3) recombinant CMTR2 sourcing (currently, it is only expressed by mammalian cells and only used to synthesize small-scale Cap2 RNA for biochemical assays). An alternative is to ligate a capped, chemically defined 5′-leader onto an IVT body using T4 RNA ligase 1, but its integration into standard mRNA manufacturing is difficult: (1) 5′-leader synthesis is challenging; (2) beyond ligation, two additional enzymatic steps are required to convert the 5′-ppp on the body to 5′-p prior to ligation; and (3) purification is not straightforward to remove excess 5′-leader and unligated body.

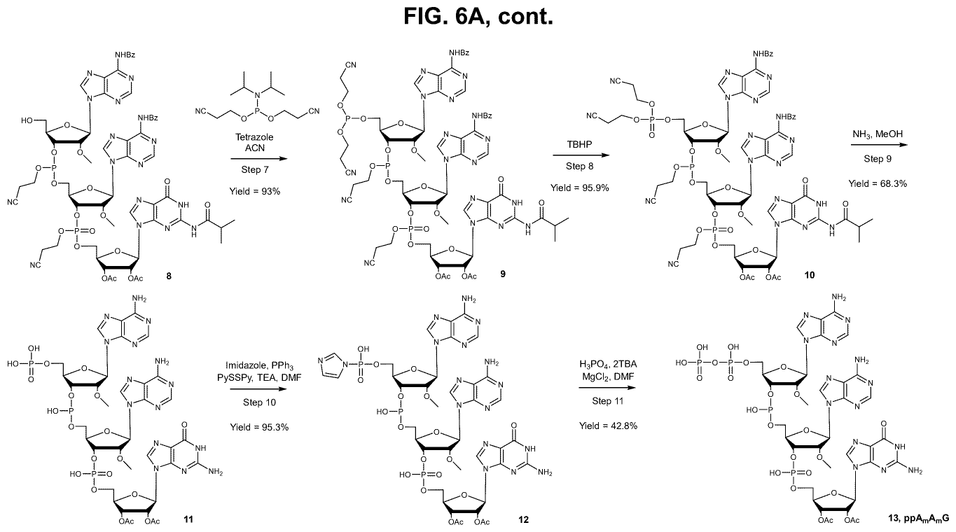

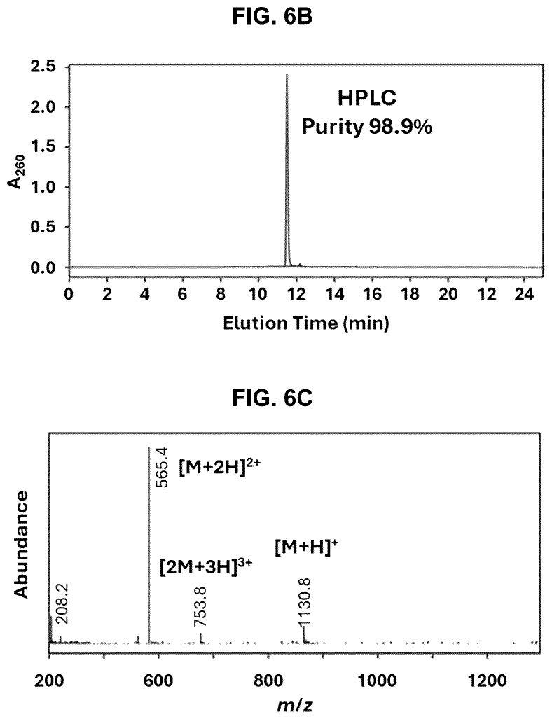

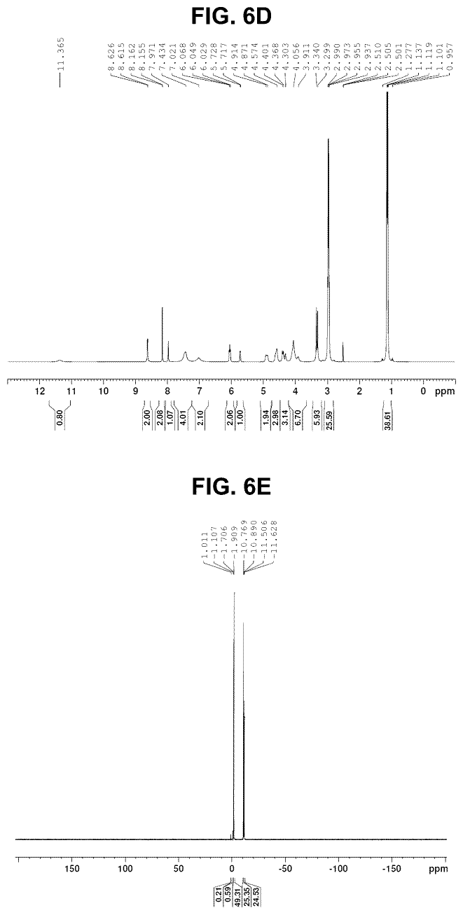

Therefore, a hybrid (co-transcriptional+enzymatic) method was developed to synthesize Cap2 mRNA (FIG. 5D). This is primed with in vitro transcription (IVT) using a cap-precursor trinucleotide (a “pre-capping reagent”), ppAₘAₘG (where ppAₘ corresponds to X in FIG. 5B) to obtain a pre-capped mRNA with 2′-O-methyl groups already at N1 and N2; then a m⁷G cap is installed enzymatically with VCE to generate Cap2 mRNA. This strategy decouples the installation of the 2′-O-methyl groups (via the co-transcriptional pre-capping reagent) from installation of m⁷G (leveraging the proven efficiency of VCE). Compared with the co-transcriptional CleanCap®, this pre-capping method features a simpler synthetic route (FIG. 6A) and avoids potential m⁷G stability issues (depurination and ring-opening). Compared with enzymatic capping, our method does not need the 2′-OMTase. The workflow is readily compatible with the established standard mRNA workflows. Further, because VCE can accommodate a range of modified GTP analogs, m⁷G variants (e.g., 3′-O-methyl-GTP leads to mCap2 containing a 3′-O-methyl on m⁷G; FIG. 5D) can be readily accessible without needing to synthesize new CleanCap®-like initiators.

Enabling IFN-Resilient mRNA Translation for Enhancing TCR-T

The new Cap2 synthesis method will enable unambiguous, side-by-side comparisons of Cap2 vs. Cap1 across diverse cell types and conditions, closing a core knowledge gap (and controversy) in mRNA translation. The results indicate that Cap2 mRNA maintains protein expression under type-I IFN, whereas matched Cap1 mRNA significantly declines, consistent with Cap2's reduced engagement of RIG-I/IFIT1. This provides an avenue to retain self-adjuvancy of unmodified RNA while preserving antigen supply for APC presentation. To our knowledge, no peer-reviewed study has yet demonstrated an mRNA vaccine boosting TCR-T (a SITC 2024 abstract reported mRNA vaccination enhancing TCR-transgenic T cell therapy in mice. Nonetheless, the principle that vaccination can deliver the full set of stimulatory signals to support adoptive T cells is well established: (1) DC vaccination (ex vivo antigen-loaded, matured DCs infused back to patients) and peptide-based vaccines could boost TCR-T; and (2) mRNA vaccines elicit potent CD8⁺ responses, including in combination settings (e.g., PD-1 inhibitor). Accordingly, our work is feasible, and novel in bringing a manufacturable Cap2 mRNA platform to the TCR-T setting, directly testing whether Cap2's IFN resilience could enable a more potent, antigen-matched TCR-T booster.

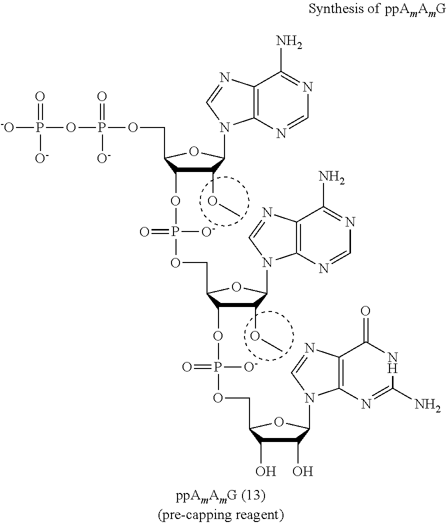

A trinucleotide pre-capping reagent, ppAₘAₘG was successfully produced (13), via a synthetic route based on solution-phase phosphoramidite chemistry (FIG. 6A). The final product 13 was obtained at high purity (98.9% by HPLC; 91.2% by ³¹P NMR) and was characterized by HPLC (FIG. 6B) and high-resolution mass spectrometry (HRMS; FIG. 6C). ¹H and ³¹P NMR spectra of 13 are shown in FIG. 6D-E, respectfully.

mRNA Caping

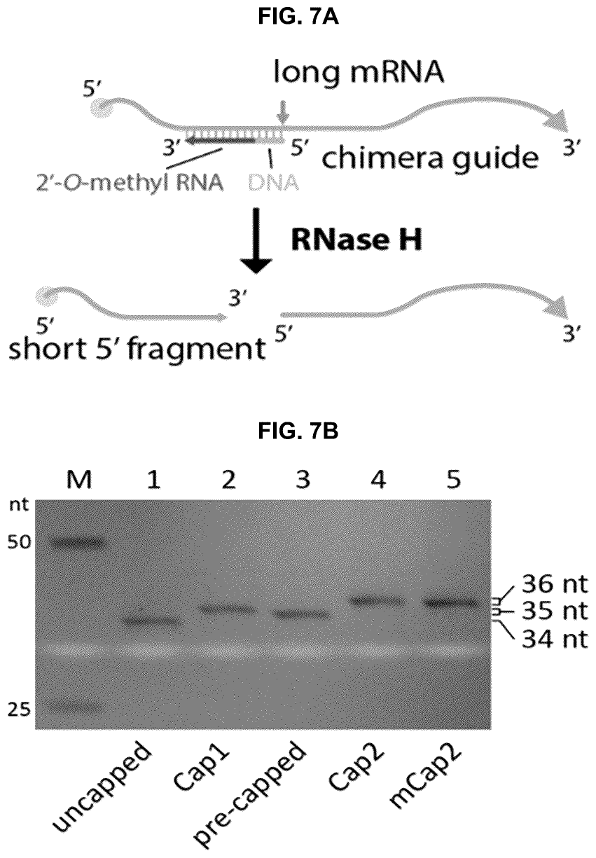

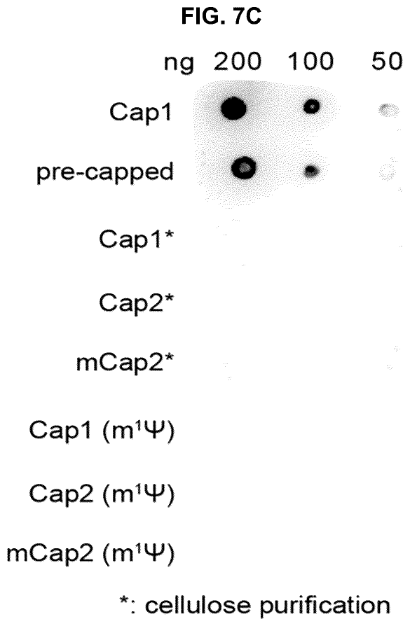

Using ppAₘAₘG, eGFP-encoding mRNAs (SEQ ID NO: 3; Table 2) were prepared bearing Cap2 and mCap2 via hybrid capping (FIG. 5D) and a Cap1 mRNA using CleanCap® (FIG. 5A). To assess the capping efficiency on these long mRNA molecules, a 2′-O-methyl RNA-DNA chimera guide strand (SEQ ID NO: 4; Table 2) was employed for site-specific RNase H-mediated cleavage (FIG. 7A) and the capping efficiency was determined by analyzing the released short 5′-fragments via denaturing polyacrylamide gel electrophoresis (PAGE), which provides clear resolution of expected mass shifts from each cap state (FIG. 7B). Results indicate near-quantitative capping for (m)Cap2 transcript. Next the level of dsRNA contaminants was evaluated by immunodot-blot assays (FIG. 7C) using the dsRNA-specific J2 mAb (monoclonal antibody), which has been validated for both unmodified and modified dsRNA. Since optimized in vitro transcription conditions were used, m14J-modified mRNA preparation showed no detectable dsRNA; trace dsRNA in unmodified mRNA preparations was readily removed by cellulose purification. While 12023 explicitly measured dsRNA, D2022 did not, raising another quality-control concern for D2022.

TABLE 2
Nucleotide Sequences

| | | SEQ ID |
|---|---|---|
| Name | Nucleotide Sequences (5′→3′) | NO |
| eGFP mRNA | GAGAAUAAACUAGUAUUCUUCUGGUCCCCACAGACUCAGAGAGAACCCG | 3 |
| | CCACCAUGGUGAGCAAGGGCGAGGAGCUGUUCACCGGGGUGGUGCCCAU | |
| | CCUGGUCGAGCUGGACGGCGACGUAAACGGCCACAAGUUCAGCGUGUCC | |
| | GGCGAGGGCGAGGGCGAUGCCACCUACGGCAAGCUGACCCUGAAGUUCA | |
| | UCUGCACCACCGGCAAGCUGCCCGUGCCCUGGCCCACCCUCGUGACCAC | |
| | CCUGACCUACGGCGUGCAGUGCUUCAGCCGCUACCCCGACCACAUGAAG | |
| | CAGCACGACUUCUUCAAGUCCGCCAUGCCCGAAGGCUACGUCCAGGAGC | |
| | GCACCAUCUUCUUCAAGGACGACGGCAACUACAAGACCCGCGCCGAGGU | |
| | GAAGUUCGAGGGCGACACCCUGGUGAACCGCAUCGAGCUGAAGGGCAUC | |
| | GACUUCAAGGAGGACGGCAACAUCCUGGGGCACAAGCUGGAGUACAACU | |
| | ACAACAGCCACAACGUCUAUAUCAUGGCCGACAAGCAGAAGAACGGCAU | |
| | CAAGGUGAACUUCAAGAUCCGCCACAACAUCGAGGACGGCAGCGUGCAG | |
| | CUCGCCGACCACUACCAGCAGAACACCCCCAUCGGCGACGGCCCCGUGC | |
| | UGCUGCCCGACAACCACUACCUGAGCACCCAGUCCGCCCUGAGCAAAGA | |
| | CCCCAACGAGAAGCGCGAUCACAUGGUCCUGCUGGAGUUCGUGACCGCC | |
| | GCCGGGAUCACUCUCGGCAUGGACGAGCUGUACAAGUAAUAGCUCUGCU | |
| | AGUUCCAGACACCUCCCAAGCACGCAGCAAUGCAGCUCAAAACGCUUAG | |
| | CCUAGCCACACCCCCACGGGAAACAGCAGUGAUUAACCUUUAGCAAUAA | |
| | ACGAAAGUUUAACUAAGCUAUACUAACCCCAGGGUUGGUCAAUUUCGUG | |
| | CCAGCCACACCCUGGAGCUAGCAAAAAAAAAAAAAAAAAAAAAAAAAAA | |
| | AAAAAAAAAAAAAAAAAAAAAAAAAAAAAAAAAAAAAAAAAAAAAAAAA | |
| | AAAA | |
| 2′-O-methyl | GTCTₘGₘUₘGₘGₘGₘGₘAₘCₘCₘA | 4 |
| RNA-DNA | | |
| Chimeric | | |
| Guide Strand | | |

For SEQ ID NO: 4, this nucleotide is a DNA/RNA chimera where the first four nucleotides, GTCT, are deoxyribonuclotides and the remainder of the nucleotide (italicized) are 2′-O-methyl-ribonucleotides. ₘN indicates a 2′-O-methyl ribonucleotide on the nucleotide preceeded by the ₘ. For example, for residue 6, ₘG, indicates a 2′-O-methyl guanosine ribonucleotide.

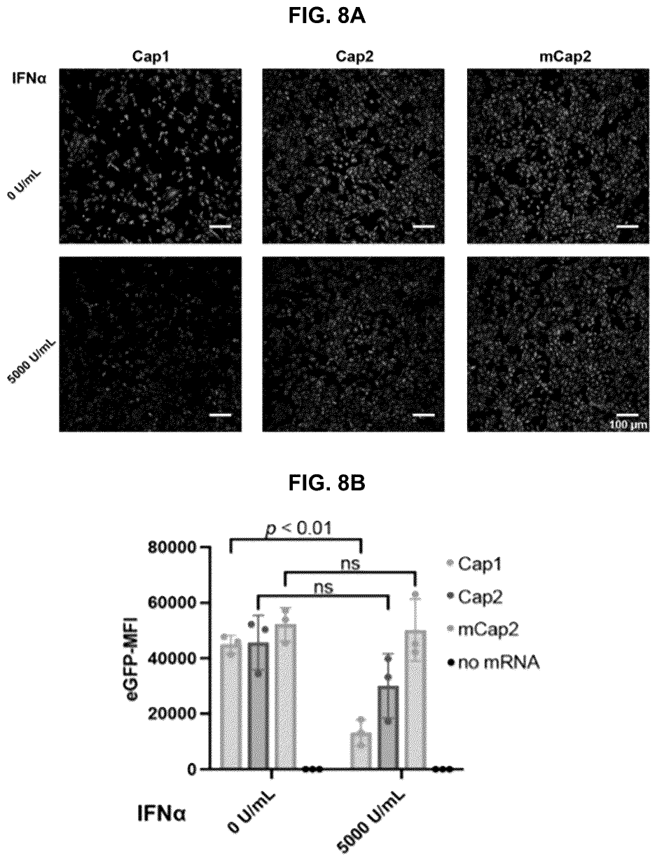

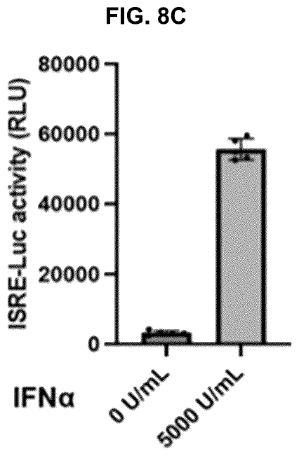

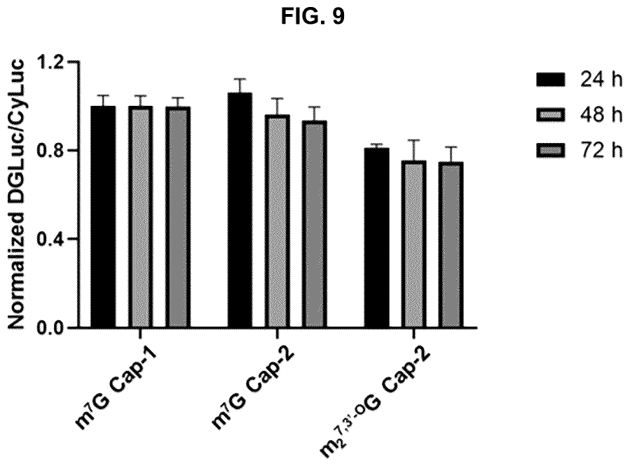

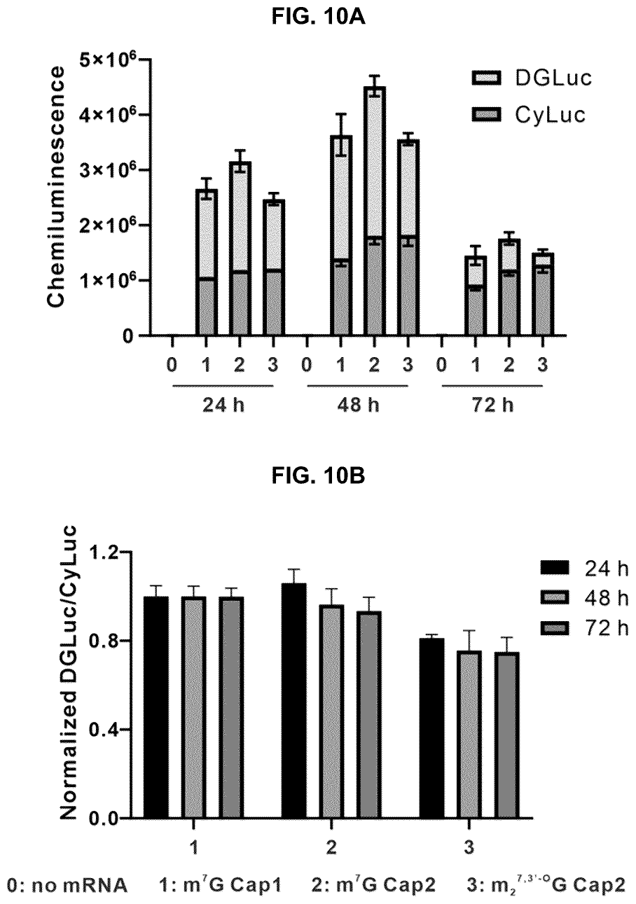

Unmodified eGFP mRNAs capped with Cap1, Cap2 and mCap2 were transfected into A549-Dual cells (InvivoGen), which are engineered to secrete luciferase under ISG54/ISRE promoter to report IFN response. At baseline, mRNAs with Cap1, Cap2 and mCap2 exhibited similar translations (FIG. 8A-B), in contrast to both D2022 and 12023. Strikingly, upon IFNα pretreatment, Cap2 and mCap2 maintained translation, whereas Cap1 decreased to ~⅓ of baseline. As expected, A549-Dual cells were responsive to IFNα treatment (FIG. 8C). To corroborate the discrepancies from D2022 and 12023, m¹ψ-modified mRNA was tested in A549-Dual, and both unmodified and m¹ψ-modified mRNAs in HeLa cells (data not shown). Under unstimulated conditions, no significant translation differences were observed across these three caps. The 12023 workflow may have inadvertently introduced factors inducing cellular stress or innate activation, which could favor Cap2 over Cap1.

Benchmark Cap2 mRNA Translation Across Additional Human Cell Lines

Additional experiments will compare Cap2 vs. Cap1 translation across different human cell lines. Human cell lines are the focus because RNA sensing by IFIT family proteins and other ISGs is species-divergent: e.g., murine IFITs differ in number and binding specificity from human IFITs43. The panel will include A549 and A549-Dual (lung epithelial), HeLa (epithelial, readily transfectable), HEK293T (low basal RIG-I, useful for low-innate baselines) and THP-1 (monocytic). Because native THP-1 monocytes display robust TLR8 but weak TLR7, THP-1 derivatives engineered to express TLR7 orTLR8 (e.g., THP1-Dual hTLR7 and THP1-Dual hTLR8; from InvivoGen) will be incorporated to model endosomal RNA sensing, allowing the evaluation of translation under physiologic TLR7/8 engagement by using unmodified mRNA as an intrinsic stimulus to induce interferon stress without exogenous IFNα.

Delivery conditions will be screened to avoid reagent bias. Baseline studies will use lipofection with Lipofectamine MessengerMAX™ transfection reagent (Thermo Fisher), followed by RiboJuice™ transfection reagent (MilliporeSigma) and JetMessenger® (Sartorious) transfection reagent. Each mRNA lot (Cap1, Cap2, mCap2) will be produced under identical in vitro transcription and purification conditions, with cap mapping and dsRNA detection to ensure that any differences reflect the cap state. To reduce experimental variability and obtain precise estimates, dual-reporter systems will be tested (e.g., eGFP/mCherry, GLuc/CLuc), in which the variable test mRNA will be co-delivered with a fixed normalizer mRNA. In A549- and THP1-Dual, interferon and inflammatory pathway activation will be recorded via secreted ISRE and NF-κB reporters; in all cell lines, ISG induction (e.g., IFITs, OAS1, PKR) will be measured by RT-qPCR, and select cytokines (e.g., IFNα/p, TNF-α, IL-6) by ELISA (enzyme-linked immunosorbent assay). Experimental conditions will span basal states, exogenous IFNα pretreatment, and PRR-driven states created either with canonical agonists (e.g., poly(I:C) for TLR3) or with unmodified mRNAs in the TLR7/8-engineered THP-1 cell lines.

Evaluate Cap2 mRNAs with N⁶-methyl on Adenosine (m⁶Am) at N1

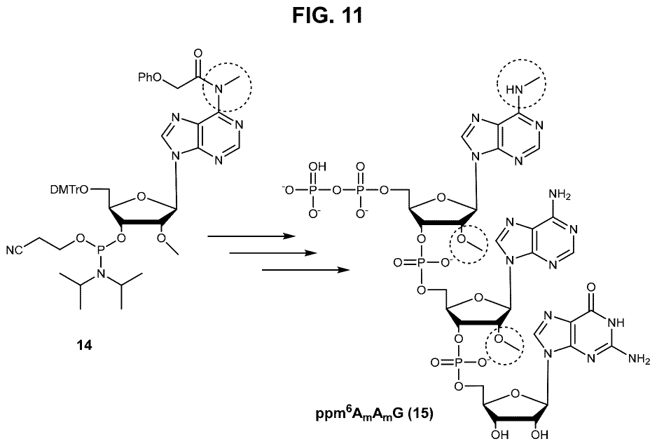

Because m⁶Am at N1 position has been shown to enhance translation for Cap1 transcripts (e.g., TriLink's CleanCap M6), whereas D2022 observed that adding m⁶Am to Cap2 mostly decreased translation in tested cell lines, motivating us to determine if m⁶Am-modified Cap2 (Cap2ₘ, Cap2 with m⁷Am at N1; ₘCap2ₘ, Cap2ₘ with a 3′-O-methyl on m⁷G) augments or compromises mRNA translation. The corresponding pre-capping trinucleotide ppm⁶AₘAₘG (FIG. 11) will be synthesized by substituting the adenosine building block (compound 2) in Step 4 of FIG. 6A with m⁶Am phosphoramidite (compound 14, available from Glen Research). The resulting reagent will then be used in the same hybrid workflow (FIG. 5D) to generate Cap2ₘ and ₘCap2ₘmRNAs.

What is claimed:

1. A method of synthesizing a Cap-2 mRNA molecule, comprising:
co-transcriptionally installing a priming trinucleotide ppAₘAₘG at a 5′-terminus of an mRNA molecule during an in vitro transcription reaction, wherein Aₘ denotes 2′-O-methyladenosine; and
adding m⁷G to the ppAₘAₘG using a vaccinia capping enzyme to yield a Cap-2 mRNA molecule comprising m⁷GpppAₘAₘG at the 5′-terminus.

2. The method of claim 1, wherein the in vitro transcription reaction comprises a T7 RNA polymerase and a DNA template comprising a T7 promoter sequence.

3. The method of claim 2, wherein the DNA template further comprises a poly(A) encoding region.

4. The method of claim 2, wherein the DNA template comprises a C+1T mutation on a template strand to initiate transcription with adenosine at a +1 position.

5. The method of claim 1, further comprising purifying the mRNA molecule by lithium chloride precipitation prior to adding the m⁷G using the vaccinia capping enzyme.

6. The method of claim 1, wherein the vaccinia capping enzyme catalyzes:
removal of a terminal phosphate from a 5′-diphosphate terminus of the mRNA molecule;
transfer of a guanosine monophosphate moiety to a resulting 5′-monophosphate terminus; and
methylation of an N7 position of a guanine base to form the m⁷GpppAₘAₘG structure.

7. The method of claim 1, wherein the adding m⁷G comprises contacting the mRNA molecule with the vaccinia capping enzyme in the presence of S-adenosylmethionine and guanosine triphosphate.

8. The method of claim 7, wherein the guanosine triphosphate is 3′-O-methylguanosine triphosphate, and wherein the Cap-2 mRNA molecule comprises m₂^{7,3′-}OGpppAₘAₘG at the 5′-terminus.

9. The method of claim 1, wherein a capping efficiency of the Cap-2 mRNA molecule is at least 95%.

10. The method of claim 9, wherein the capping efficiency of the Cap-2 mRNA molecule is at least 99%.

11. The method of claim 1, wherein the in vitro transcription reaction further comprises N1-methylpseudouridine triphosphate, and wherein the Cap-2 mRNA molecule comprises N1-methylpseudouridine nucleobases.

12. The method of claim 1, further comprising purifying the Cap-2 mRNA molecule to remove the vaccinia capping enzyme and salts following the adding m⁷G.

13. The method of claim 12, further comprising removing double-stranded RNA contaminants from the Cap-2 mRNA molecule by cellulose purification.

14. The method of claim 1, wherein the priming trinucleotide ppAₘAₘG has a purity of at least 95%.

15. The method of claim 14, wherein the priming trinucleotide ppAₘAₘG has a purity of at least 98%.

16. The method of claim 1, wherein the Cap-2 mRNA molecule encodes a therapeutic protein or an antigen.

17. The method of claim 16, further comprising formulating the Cap-2 mRNA molecule in a lipid nanoparticle.

18. A trinucleotide compound having a structure of ppAₘAₘG, wherein pp denotes a diphosphate moiety at a 5′-terminus, Aₘ denotes 2′-O-methyladenosine, and G denotes guanosine, or a pharmaceutically acceptable salt thereof.

19. The trinucleotide compound of claim 18, wherein the trinucleotide compound has a purity of at least 95% as determined by high-performance liquid chromatography.

20. The trinucleotide compound of claim 18, wherein the guanosine comprises a 2′-hydroxyl group and a 3′-hydroxyl group on a ribose sugar.


SEQUENCE LISTING (4 sequences)

SEQ ID NO: 1 - DNA, 23 bp, synthetic construct
  source 1..23: mol_type = other DNA
gttctaatacgactcactataag

SEQ ID NO: 2 - DNA, 23 bp, synthetic construct
  source 1..23: mol_type = other DNA
caagattatgctgagtgatattc

SEQ ID NO: 3 - RNA, 1033 nt, synthetic construct
  source 1..1033: mol_type = other RNA
gagaataaactagtattcttctggtccccacagactcagagagaacccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 14 bp, synthetic construct
  source 1..14: mol_type = other DNA
gtctgtggggacca